(19) United States
(12) Patent Application Publication
(10) Pub. No.: US 2005/0019806 A1
(43) Pub. Date: Jan. 27, 2005

(54) Nucleic acids and polypeptides required for cell survival in the absence of Rb

(76) Inventors: H. Robert Horvitz, Auburndale, MA (US); Michael Hurwitz, Cambridge, MA (US)

Correspondence Address: CLARK & ELBING LLP, 101 FEDERAL STREET, BOSTON, MA, 02110, US

(21) Appl. No.: 10/879,330

(22) Filed: Jun. 29, 2004

Related U.S. Application Data
(60) Provisional application No. 60/484,194, filed on Jun. 30, 2003.
(60) Provisional application No. 60/510,096, filed on Oct. 9, 2003.

(51) Int. Cl. C12Q 1/68; G01N 33/574

(52) U.S. Cl. 435/6; 435/7.23

(57) ABSTRACT

In general, the invention features nucleic acid and amino acid sequences required for cell survival in the absence of Rb. These sequences may be used in screening methods for identifying therapeutics for neoplasia treatment.

### CROSS-REFERENCE TO RELATED APPLICATIONS

This application claims the benefit of U.S. provisional application Nos. 60/484,194, and 60/510,096, filed on Jun. 30, 2003 and Oct. 9, 2003, respectively, each of which is hereby incorporated by reference.

### STATEMENT AS TO FEDERALLY SPONSORED RESEARCH

The present research was supported by grants from the National Institutes of Health (NIH 5 T32 CA71345, NIH RO1 GM24663-24, GM24663-25, GM24663-26). The U.S. government has certain rights to this invention.

### BACKGROUND OF THE INVENTION

In general, the invention features nucleic acid and amino acid sequences required for cell survival in the absence of Rb. These sequences may be used in screening methods for identifying therapeutics for neoplasia treatment.

A molecular pathway that contains the protein Rb, the retinoblastoma protein, is inactivated in almost all carcinomas, which comprise ˜85% of all cancers. Data from cell lines, mice, and analysis of tumor tissues indicate that inactivation of the Rb pathway may be a prerequisite for cancer development and progression.

Molecular data from mice, human cell lines, and C. elegans indicate that the Rb pathway is well conserved between the three species. C. elegans contains a single Rb family protein called lin-35 Rb. lin-35 Rb has been shown to interact genetically with the worm orthologs of mammalian Rb-interacting proteins: E2F, DP, histone deacetylase (HDAC), and RbAp48. The C. elegans orthologs of these genes are efl-1, dpl-1, hda-1, lin-53, respectively. These genes, as well as lin-35 Rb, are members of a set of genes called the synMuv B genes that oppose a Ras-MAP Kinase pathway in C. elegans. In addition to these genetic interactions, lin-35 Rb has been shown to interact physically with EFL-1 and DPL-1 to repress transcription. lin-35 Rb has also been shown to physically interact with lin-53 RbAp48 and hda-1 HDAC. Upstream elements of the Rb pathway in worms are also conserved. Inactivation of the cyclin D homolog, cyd-1, and the CDK4 ortholog, cdk-4, each leads to G1 arrest in C. elegans larvae. This arrest can be partially rescued by lin-35 Rb inactivation, consistent with the role of Rb in mammals as a protein which blocks cell-cycle progression until it is suppressed by cyclin D/CDK4. Also analogous to mammals, cki-1, a Cip/Kip CKI, acts together and in parallel with lin-35 Rb to block cell cycle progression. A gene homologous to INK4 family genes exists in worms but has not been studied.

In contrast to Rb^{-/-} mice, which die as early embryos, homozgous lin-35 Rb C. elegans appear phenotypically normal, but lay fewer eggs and develop more slowly than their wild-type counterparts. Despite the differences in severity of the effects of Rb inactivation on the viability of mice and worms, the function of Rb in the two organisms is quite similar. In both mice and worms, Rb antagonizes the Ras pathway and affects cell cycle progression and development.

A need exists for improved methods for inhibiting the proliferation of neoplastic cells in a subject. Given the similarities that exist between the nematode and mammalian Rb pathways, C. elegans provides an efficient, inexpensive, and facile screening tool to identify novel clinical targets and chemotherapeutics useful in the treatment of neoplasias.

### SUMMARY OF THE INVENTION

As described below, the present invention features compositions and methods relating to nucleic acid and amino acid sequences required for cell survival in the absence of Rb. Specifically featured are inhibitory nucleic acids that target the human and nematode sequences identified in Tables 1 and 2. The sequences listed in Tables 1 and 2 may be used in either purified or naturally occurring contexts in screening methods for identifying therapeutics and in methods for the treatment of a neoplasia. Table 1 identifies the target genes by C. elegans cosmid name and open reading frame number. Nucleic acid and polypeptide sequence information is available at wormbase (www.wormbase.org), a central repository of data on C. elegans. The human genes are identified by gene name and their encoded proteins are identified in Table 2 by Genbank Accession number. Nucleic acid and polypeptide sequence information is available at the National Center for Biotechnology Information, National Library of Medicine, National Institutes of Health website, at http://www.ncbi.nlm.nih.gov/entrez.

In one aspect, the invention generally provides a method for identifying a gene required for survival in a nematode having a mutation (e.g., a deletion, a point mutation, or an insertion) in Rb or in a component of an Rb pathway (e.g., efl-1, dpl-1, hda-1, and lin-53). The method involves providing a nematode having a mutation in Rb or in a component of an Rb pathway; contacting the nematode with an inhibitory nucleic acid; and comparing the phenotype (e.g., sterility, embryonic lethality, larval lethality, or larval growth arrest prior to L3) of the nematode contacted with the inhibitory nucleic acid with the phenotype of a control nematode not contacted with the inhibitory nucleic acid, where an alteration in the phenotype of the nematode identifies the gene as a gene that is required for survival in a nematode having a mutation in Rb or in a component of an Rb pathway.

In another aspect, the invention provides a method for identifying a gene required for cell survival in a mammalian cell (e.g., a human or neoplastic cell) having a mutation in Rb or in a component of an Rb pathway. The method involves providing a mammalian cell having a mutation in Rb or in a component of an Rb pathway; contacting the cell with an inhibitory nucleic acid; and comparing the survival of the cell contacted with the inhibitory nucleic acid with the survival of a control cell not contacted with the inhibitory nucleic acid, where a decrease (10%, 25%, 50%, 75%, 100%) in the survival of the cell identifies the gene as a gene that is required for survival in a nematode having a mutation in Rb or in a component of an Rb pathway. In one embodiment, the cell has a mutation in Dp, E2F, or histone deacetylase.

In another aspect, the invention provides a method for identifying a candidate compound for the treatment or prevention of neoplasia. The method involves providing a cell that expresses a nucleic acid required for cell survival in the absence of Rb; contacting the cell with a candidate compound; and comparing the expression of the nucleic acid molecule in the cell contacted with the candidate compound with the expression of the nucleic acid molecule in a control cell not contacted with the candidate compound, where a decrease in the expression identifies the candidate compound as a candidate compound that treats a neoplasia. In one embodiment, the cell is a nematode cell. In another embodiment, the cell is in a nematode. In another embodiment, the cell is a mammalian, human, or neoplastic cell. In another embodiment, the cell comprises a mutation in Rb or in a component of an Rb pathway (e.g., Dp, E2F, or histone deacetylase). In another embodiment, the method further comprises detecting a decrease in cell survival. In a related embodiment, the detecting identifies an increase in apoptosis. In yet another embodiment, the expression is detected by measuring a decrease in transcription. In another embodiment, the expression is detected by measuring a decrease in translation.

In another aspect, the invention features a method for identifying a candidate compound for the treatment or prevention of a neoplasia. The method involves providing a cell expressing a gene required for cell survival in the absence of Rb and having a mutation in Rb or in a component of an Rb pathway; contacting the cell with an inhibitory nucleic acid; and detecting a decrease in survival of the cell contacted with the inhibitory nucleic acid relative to the survival of a control cell not contacted with the inhibitory nucleic acid, where a decrease in survival in the contacted cell identifies the inhibitory nucleic acid as an inhibitory nucleic acid for the treatment or prevention of neoplasia. In one embodiment, the cell is in a nematode. In another embodiment, the cell is a mammalian (e.g., rodent or human) cell.

In additional embodiments of any of the previous aspects, the inhibitory nucleic acid (e.g., siRNA, shRNA, antisense RNA, dsRNA) hybridizes (or contains an antisense strand that hybridizes) under high stringency conditions with at least a portion of any one of the nucleic acid molecules required for cell survival in the absence of Rb (e.g., those nucleic acid molecules listed in Tables 1 or 2). In other embodiments, the method detects an increase in apoptosis. In other embodiments, the cell has a mutation in Dp, E2F, or histone deacetylase.

In another aspect, the invention features a method for identifying a candidate compound for the treatment or prevention of neoplasia. The method involves providing a cell expressing a nucleic acid required for survival in a cell lacking Rb; contacting the cell with a candidate compound; and comparing the biological activity of the polypeptide in the cell contacted with the candidate compound to a control cell not contacted with the candidate compound, where a decrease (e.g., 10%, 25%, 50%, 75%, or 100%) in the biological activity of the polypeptide identifies the candidate compound as a candidate compound for the treatment or prevention of a neoplasia. In one embodiment, the cell is a nematode cell or is in a nematode. In another embodiment, the cell is a mammalian cell (e.g., rodent or human). In another embodiment, the biological activity is monitored with an enzymatic assay, an immunological assay, or an assay for cell survival. In another embodiment, the cell comprises a mutation in Rb or in a component of an Rb pathway. In yet another embodiment, the cell is in a nematode having a mutation in Rb and the biological activity is monitored by detecting sterility, embryonic lethality, larval lethality, or larval growth arrest prior to L3. In one embodiment, the comparing detects an increase in apoptosis. In another embodiment, the cell has a mutation in Dp, E2F, or histone deacetylase.

In a related aspect, the invention features a method of identifying a candidate compound for the treatment or prevention of neoplasia. The method involves contacting a cell with an candidate compound, where the cell contains a nucleic acid that is required for cell survival in the absence of Rb, and wherein the nucleic acid is fused to a detectable reporter gene; detecting the expression of the reporter gene; and comparing the reporter gene expression in the cell contacted with the candidate compound with a control cell not contacted with the candidate compound, where a decrease in the expression of the reporter gene identifies the candidate compound as a compound for the treatment or prevention of neoplasia. In one embodiment, the decrease is a decrease of at least 10%, 20%, or 30%, more preferably of 40%, 50%, 75%, 85%, or most preferably of 95% in the level of expression of the reporter gene relative to the level of expression in a control cell not contacted with the candidate compound.

In another related aspect, the invention features a method for identifying a candidate compound for the treatment or prevention of neoplasia, which includes contacting a polypeptide required for cell survival in a cell lacking Rb with a candidate compound; and detecting binding of the candidate compound to the polypeptide, where the binding identifies the candidate compound as a candidate compound for the treatment or prevention of neoplasia.

In another aspect, the invention features an isolated nucleic acid inhibitor that contains at least a portion of a naturally occurring nucleic acid molecule of an organism, or its complement, required for cell survival in a cell lacking Rb, where the nucleic acid inhibitor decreases expression of the nucleic acid molecule in a cell of an organism. In one embodiment, the RNA is a double stranded RNA molecule that decreases expression in the organism by at least 10%, 20%, or 30%, more preferably by at least 40%, 50%, 75%, 85%, or most preferably by at least 95%. In another embodiment, the RNA molecule is an antisense nucleic acid molecule that is complementary to at least 6, 10, 20, 30, 40, 50, 60, or 100 nucleotides of the nucleic acid molecule. In another embodiment, the RNA molecule is an siRNA molecule that comprises at least 5, 10, 15, 17, 18, 19, 20, 21, 22, 23, 24, 25, 30, or 35 nucleic acids of the nucleic acid molecule and decreases expression in the organism by at least 10%, 20%, or 30%, more preferably of 40%, 50%, 75%, 85%, or most preferably of 95%.

In another aspect, the invention features a vector encoding a nucleic acid inhibitor of any previous aspect positioned for expression.

In yet another aspect, the invention features a host cell containing the vector of the previous aspect.

In another aspect, the invention features an isolated nucleic acid inhibitor containing at least a portion of a naturally occurring nucleic acid molecule of an organism, or its complement, required for cell survival in a mammalian cell lacking Rb, where the nucleic acid inhibitor decreases expression of the nucleic acid molecule in a mammalian cell. In one embodiment, the RNA is a double stranded RNA molecule that decreases expression in the organism by at least 10%, 20%, or 30%, more preferably by 40%, 50%, 75%, 85%, or most preferably by 95%. In another embodiment, the RNA molecule is an antisense nucleic acid molecule that is complementary to at least 6, 10, 20, 30, 40, 50, 60, or 100 nucleotides of the nucleic acid molecule and decreases expression in the organism by at least 10%, 20%, or 30%, more preferably by 40%, 50%, 75%, 85%, or most preferably by 95%. In another embodiment, the RNA molecule is an siRNA molecule that comprises at least 5, 10, 15, 17, 18, 19, 20, 21, 22, 23, 24, 25, 30, or 35 nucleic acids of the nucleic acid molecule and decreases expression in the organism by at least 10%, 20%, or 30%, more preferably of 40%, 50%, 75%, 85%, or most preferably of 95%.

In a related aspect, the vector contains the nucleic acid inhibitor of the previous aspect positioned for expression.

In another related aspect, the invention features a host cell containing the vector of the previous aspect.

In another aspect, the invention features a method for treating neoplasia in a subject, the method involves contacting a cell of the subject (e.g., a mammal, such as a human) with a nucleic acid inhibitor of any of the previous aspects in an amount sufficient to treat neoplasia in the subject. In one embodiment, the nucleic acid inhibitor is a double stranded RNA molecule that comprises at least 20, 30, 40, 50, 75, 100, or 125 nucleic acids of a nucleic acid molecule required for cell survival in the absence of Rb and is capable of hybridizing to the nucleic acid molecule under high stringency conditions, and is capable of decreasing expression of the nucleic acid molecule in the organism with which it shares identity by at least 10%, 20%, or 30%, more preferably of 40%, 50%, 75%, 85%, or most preferably of 95%. In another embodiment, the nucleic acid inhibitor is an antisense nucleic acid molecule that is complementary to at least 6, 10, 20, 30, 40, 50, 60, or 100 nucleotides of a nucleic acid molecule required for cell survival in the absence of Rb, and is capable of hybridizing to the nucleic acid molecule under high stringency conditions and is capable of decreasing expression of the nucleic acid molecule by at least 10%, 20%, or 30%, more preferably of 40%, 50%, 75%, 85%, or most preferably of 95%. In another embodiment, the nucleic acid inhibitor is an siRNA molecule that comprises at least 5, 10, 15, 17, 18, 19, 20, 21, 22, 23, 24, 25, 30, or 35 nucleic acids of a nucleic acid required for cell survival in the absence of Rb, and is capable of hybridizing to the nucleic acid molecule under high stringency conditions and is capable of decreasing expression by at least 10%, 20%, or 30%, more preferably of 40%, 50%, 75%, 85%, or most preferably of 95% from the nucleic acid molecule with which it shares identity

In another aspect, the invention features a pharmaceutical composition including a pharmaceutical excipient and a nucleic acid inhibitor of any of the previous aspects, where the nucleic acid inhibitor is present in an amount sufficient for the treatment or prevention of neoplasia in subject.

In a related aspect, the invention features a method of treating or preventing a neoplastic disease, The method involves administering to a subject in need of such treatment an effective amount of a pharmaceutical composition containing a nucleic acid inhibitor of a gene required for cell survival in the absence of Rb.

In another related aspect, the invention features a method of treating or preventing a neoplastic disease, the method involves administering to a patient in need of such treatment an effective amount of a pharmaceutical composition that contains a compound that inhibits the biological activity of a polypeptide required for cell survival in the absence of Rb.

In another aspect, the invention features a method of treating or preventing a neoplastic disease, the method involves administering to a patient in need of such treatment an effective amount of a pharmaceutical composition that decreases expression of a gene required for cell survival in the absence of Rb.

In another aspect, the invention features a collection of primer sets, each of the primer sets contains at least two primers that bind to a nucleic acid molecule required for cell survival in the absence of Rb under high stringency conditions, where the collection contains at least two different primer sets.

In another related aspect, the invention features a purified nucleic acid library containing at least two nucleic acid molecules required for cell survival in the absence of Rb, where at least 50%, 60%, 70%, 80%, 90%, or 95% of the nucleic acid molecules present in the library are required for cell survival in the absence of Rb. In one embodiment, the nucleic acid molecules in the library are carried in a vector. In another embodiment, each of the nucleic acid molecules is fused to a reporter gene.

In another aspect, the invention features a method of identifying a candidate compound for the treatment or prevention of neoplasia, the method involves contacting a cell containing a member of the library of the previous aspect; measuring the expression of the reporter gene; and comparing the level of reporter gene expression in the cell contacted with the candidate compound with the level in a control cell not contacted with the candidate compound, where a decrease in the level of the reporter gene expression identifies the candidate compound as a candidate compound for the treatment or prevention of neoplasia.

In another aspect, the invention features a microarray containing at least two nucleic acid molecules, or fragments thereof, bound to a solid support, where at least 50%, 60%, 70%, 80%, 90%, or 95% of the nucleic acids molecules on the support are required for cell survival in the absence of Rb.

In another aspect, the invention features a method of identifying a candidate compound for the treatment or prevention of a neoplasia. The method involves contacting a cell with a candidate compound; obtaining a nucleic acid from the cell; c) contacting a microarray of the previous aspect with the nucleic acid; and d) detecting a decrease in expression level of a gene required for cell survival in the absence of Rb in a cell contacted with the candidate compound compared to a control cell, where the decrease identifies the candidate compound as a candidate compound for the treatment or prevention of a neoplasia.

In another aspect, the invention features a method of monitoring a patient diagnosed as having a neoplasia, the method involves determining the level of expression of a nucleic acid molecule or polypeptide required for cell survival in the absence of Rb in a patient sample, where a decrease in the level of expression relative to the level of expression in a control sample indicates the severity of a neoplasia in the patient. In one embodiment, the control sample is a normal patient sample. In another embodiment, the control sample is a reference sample taken from the patient. In another embodiment, the patient is being treated for a neoplasia.

In various embodiments of any of the previous aspects, the isolated nucleic acid is selected from any one of the group consisting of C43E11.10, M04F3.1, F28B3.7, C55B7.8, T21G5.3, F22D6.5, R05D11.7, C25A1.3, C44E4.4, C06A5.5, C37A2.4, T25G3.3, T05E8.3, D1081.8, B0511.6, Y105E8C.e, Y40B1B.6, ZC123.3, H06O01.3, K02B12.1, Y47H9C.7, Y65B4A_{—}182.c, C48E7.2, F14B4.3, W04A8.7, R12E2.12, Y106G6H.3, C43E11.9, T25G3.3, C03D6.8, T23D8.4, F46F11.4, B0207.6, C55B7.6, M05B5.2, F07A5.1, F27D4.1, F45H11.2, R06C1.3, W09C5.8, Y54E5A.4, R06A10.2, M01B12.3, R12E2.10, F55A12.7, C01H6.7, F30A10.6, T23D8.1, Y52B11A.9, T04D3.3, Y40B1A.4, C01A2.3, Y87G2A.s, Y6B3A.1, Y54E10_{—}156.a, K07A12.2, C43H8.3, C32E8.5, T03F1.8, F57C9.4, F28B3.1, T19B4.4, C10G11.5, T21G5.4, C30F12.4, F27D4.5, C09H6.1, C34B7.3, F30A10.10, F26E4.6, F27C1.6, C55B7.9, C36B1.8, ZK858.2, ZK39.6, K08C9.1, Y34D9A_{—}151.a, Y54E10_{—}155.c, Y54E10_{—}155.e, R119.6, C10H11.10, F48C1.4, F55A12.8, ZC308.2, ZK265.6, F30A10.9, T23D8.3, Y95D11A.a, Y87G2A.e, Y71A12B.b, Y48G8A_{—}3671.a, Y65B4A_{—}174.b, ZC123.2, R12E2.2, T19B4.5, C26C6.5, F02E9.3, F39H2.3, ZK858.7, C03D6.1, Y39G10A_{—}246.I, Y39G10A_{—}246.j, Y47G6A_{—}247.h, Y48G1A_{—}54.d, and Y54E10B_{—}159.e, or a fragment thereof, or an ortholog thereof.

In other embodiments of any of the previous aspects, the isolated nucleic acid molecule encodes a protein selected from any one of the group consisting of CDC6, ORC1L, ORC4L, RPA2, SMC1L1, SMC1L2, DBR1, FLJ10998, DDX4, DDX3, DBY, PRP4, HIPK3, HIPK2, RY1, RNMT, SSB, LOC51068, DDX33, DDX8, DDX38, CDC5L, DDX18, KIAA0601, C20ORF16, ATBF1, SEC14L2, RNAP3, RPC62, RPO1-2, POLR2B, FLJ10388, TAF1, RPL30, HSPC031, CGI-07, LOC51068, C15ORF15, EIF3S8, UBL5, FLJ10349, SLC26A2, SLC26A8b, SLC26A8a, SLC26A1a, SLC26A1b, SLC26A1c, ETFA, UBA52, RPS27A, UBB, WASF1, COX411, COX4I2, MUC1, GNAS, GNAL, ARPC5, MGC3038, AP1M1, BRD7, FLJ13441, BRD1, SACM1L, SAC2, SYNJ1, FZD1, FZD7, FZD2, KIN, PDE1A, PDE1C, PDE1B, OXAOXA1L, VPS28, ARFGEF2, GUK1, KIAA0934, MCJ, FLJ10782, MSF, BCKDHB, ZNF208, CYP2A13, CYP2A7, CYP2A6, NP_{—}115612, NP_{—}001858, KIAA0266, FLJ20045, FLJ10774, HSPC111, CGI-35, LOC51077, F36A2.12, XP_{—}293124, and LOC51605 or encodes a protein whose GenBank Accession Number is listed in Table 2.

In yet other embodiments of any of the previous aspects, a nucleic acid inhibitor (e.g., an antisense nucleic acid, dsRNA, shRNA, or siRNA, or analog thereof) targets any nucleic acid molecule selected from the group consisting of genes listed in Tables 1 or 2 (e.g., C43E11.10, M04F3.1, F28B3.7, C55B7.8, T21G5.3, F22D6.5, R05D11.7, C25A1.3, C44E4.4, C06A5.5, C37A2.4, T25G3.3, T05E8.3, D1081.8, B0511.6, Y105E8C.e, Y40B1B.6, ZC123.3, H06O01.3, K02B12.1, Y47H9C.7, Y65B4A_{—}182.c, C48E7.2, F14B4.3, W04A8.7, R12E2.12, Y106G6H.3, C43E 11.9, T25G3.3, C03D6.8, T23D8.4, F46F11.4, B0207.6, C55B7.6, M05B5.2, F07A5.1, F27D4.1, F45H11.2, R06C1.3, W09C5.8, Y54E5A.4, R06A10.2, M01B12.3, R12E2.10, F55A12.7, C01H6.7, F30A10.6, T23D8.1, Y52B11A.9, T04D3.3, Y40B1A.4, C01A2.3, Y87G2A.s, Y6B3A.1, Y54E10_{—}156.a, K07A12.2, C43H8.3, C32E8.5, T03F1.8, F57C9.4, F28B3.1, T19B4.4, C10G11.5, T21G5.4, C30F12.4, F27D4.5, C09H6.1, C34B7.3, F30A10.10, F26E4.6, F27C1.6, C55B7.9, C36B1.8, ZK858.2, ZK39.6, K08C9.1, Y34D9A_{—}151.a, Y54E10_{—}155.c, Y54E10_{—}155.e, R119.6, C10H11.10, F48C1.4, F55A12.8, ZC308.2, ZK265.6, F30A10.9, T23D8.3, Y95D11A.a, Y87G2A.e, Y71A12B.b, Y48G8A_{—}3671.a, Y65B4A_{—}174.b, ZC123.2, R12E2.2, T19B4.5, C26C6.5, F02E9.3, F39H2.3, ZK858.7, C03D6.1, Y39G10A_{—}246.I, Y39G10A_{—}246.j, Y47G6A_{—}247.h, Y48G1A_{—}54.d, and Y54E10B_{—}159.e) or an ortholog thereof.

In other embodiments of any of the previous aspects, a nucleic acid inhibitor (e.g., an antisense nucleic acid, dsRNA, shRNA, or siRNA, or analog thereof) targets any nucleic acid molecule encoding a protein selected from the group consisting of those listed in Tables 1 or 2 (e.g., CDC6, ORC1L, ORC4L, RPA2, SMC1L1, SMC1L2, DBR1, FLJ10998, DDX4, DDX3, DBY, PRP4, HIPK3, HIPK2, RY1, RNMT, SSB, LOC51068, DDX33, DDX8, DDX38, CDC5L, DDX18, KIAA0601, C20ORF16, ATBF1, SEC14L2, RNAP3, RPC62, RPO1-2, POLR2B, FLJ10388, TAF1, RPL30, HSPC031, CGI-07, LOC51068, C15ORF15, EIF3S8, UBL5, FLJ10349, SLC26A2, SLC26A8b, SLC26A8a, SLC26A1a, SLC26A1b, SLC26A1c, ETFA, UBA52, RPS27A, UBB, WASF1, COX4I1, COX4I2, MUC1, GNAS, GNAL, ARPC5, MGC3038, AP1M1, BRD7, FLJ13441, BRD1, SACM1L, SAC2, SYNJ1, FZD1, FZD2, KIN, PDE1A, PDE1C, PDE1B, OXAOXA1L, VPS28, ARFGEF2, GUK1, KIAA0934, MCJ, FLJ10782, MSF, BCKDHB, ZNF208, CYP2A13, CYP2A7, CYP2A6, NP_{—}115612, NP_{—}001858, KIAA0266, FLJ20045, FLJ10774, HSPC111, CGI-35, LOC51077, F36A2.12, XP_{—}293124, and LOC51605) or encodes a protein whose GenBank Accession Number is listed in Table 1.

By “absence of Rb” is meant a decrease in the level of functional Rb or in the activity of Rb or in the function of an Rb pathway sufficient to detect an alteration in a phenotype associated with apoptosis, cellular proliferation, or oncogenesis. Such a decrease could be a decrease of at least 10%, 25%, 50%, 75%, or even of 100% in the biological activity of Rb or of an Rb pathway. The level of functional Rb is detected by measuring the expression of an Rb protein or nucleic acid, the expression of a protein or nucleic acid component of an Rb pathway (e.g., E2F, DP, histone deacetylase (HDAC), and RbAp48), or by measuring the biological activity of Rb or of an Rb pathway. Methods for assaying the biological activity of Rb or of an Rb pathway are known in the art (e.g., Duanduan et al. J. Biol. Chem. 278:19358-19366, 2003; Harbour et al., Genes and Dev. 14:2393-2409, 2000; Lu et al., Cell 95:981-91, 1998; Ceol et al., Mol. Cell 7:461-73, 2001; Page et al., Mol. Cell 7:451-60, 2001).

By “anti-sense” is meant a nucleic acid sequence, regardless of length, that is complementary to the coding strand or mRNA of a nucleic acid sequence. Desirably the anti-sense nucleic acid is capable of decreasing the expression or biological activity of a nucleic acid or amino acid sequence. In a desirable embodiment, the decrease in expression or biological activity is at least 10%, relative to a control, more desirably 25%, and most desirably 50% or more. The anti-sense nucleic acid may contain a modified backbone, for example, phosphorothioate, phosphorodithioate, or other modified backbones known in the art, or may contain non-natural internucleoside linkages.

By “binds” is meant a compound or antibody which recognizes and binds a polypeptide of the invention, but which does not substantially recognize and bind other different molecules in a sample, for example, a biological sample, which naturally includes a polypeptide of the invention.

By “biological activity of a polypeptide required for cell survival in the absence of Rb” is meant an activity that is required for cellular function in a cell that lacks Rb or a functional Rb pathway. A decrease in the biological activity of such a polypeptide (e.g., those listed in Tables 1 or 2) is assayed by detecting sterility, embryonic lethality, larval lethality, or larval growth arrest prior to adulthood. Other biological activities, which can be assayed using methods known to the skilled artisan, are listed under the heading “Function/Identity” in Tables 1 and 2.

By “cell” is meant a single-celled organism, a cell from a multi-cellular organism, or it may be a cell contained in a multi-cellular organism.

By “collection” is meant a group having at least 2, 5, 10, 15, 20, 25, 30, 35, 40, 45, or 50 members.

By “derived from” is meant isolated from or having the sequence of a naturally-occurring sequence (e.g., a cDNA, genomic DNA, synthetic, or combination thereof). “Differentially expressed” means a difference in the expression level of a nucleic acid. This difference may be either an increase or a decrease in expression, when compared to control conditions.

By “double stranded (ds) RNA” is meant a complementary pair of sense and antisense RNAs regardless of length. In one embodiment, these dsRNAs are introduced to an individual cell, tissue, organ, or to whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The dsRNA may contain a modified backbone, for example, phosphorothioate, phosphorodithioate, or other modified backbones known in the art, or may contain non-natural internucleoside linkages.

By “fragment” is meant a portion of a protein or nucleic acid that is substantially identical to a reference protein or nucleic acid (e.g., one of those listed in Tables 1 or 2), and retains at least 50% or 75%, more preferably 80%, 90%, or 95%, or even 99% of the biological activity of the reference protein or nucleic acid using a nematode bioassay as described herein or a standard biochemical or enzymatic assay.

By “hybridize” is meant pair to form a double-stranded molecule between complementary polynucleotide sequences (e.g., genes listed in Tables 1 and 2), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “immunological assay” is meant an assay that relies on an immunological reaction, for example, antibody binding to an antigen. Examples of immunological assays include ELISAs, Western blots, immunoprecipitations, and other assays known to the skilled artisan.

By “inhibitory nucleic acid” is meant a double-stranded RNA, siRNA, shRNA, or antisense RNA, or a portion thereof, or a mimetic thereof, that when administered to a nematode, nematode cell, or mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids listed in Table 1 or 2.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed to aid in identification of the individual nucleic acid molecules or polypeptides. A microarray may include, for example, nucleic acid molecules representing all, or a subset, of the open reading frames of an organism, or of the polypeptides that those open reading frames encode. In one embodiment, the nucleic acid molecules of the array are defined as having a common region of the genome having limited homology to other regions of an organism's genome. A microarray may also be enriched for a particular type of gene. In one example, a microarray of “nucleic acid molecules required for cell survival in the absence of Rb” may be enriched for nucleic acid molecules or their encoded polypeptides so that, for example, it comprises at least 5%, 10%, 15%, 20%, 22%, 25%, 30%, 40%, 50%, 60%, 70%, 80%, 90%, 95%, 97% or even 99% nucleic acid molecule required for cell survival in the absence of Rb or their encoded polypeptides. In one example, a “microarray of nucleic acid molecules required in the absence of Rb” or their encoded polypeptides contains any one of or all of the C. elegans or mammalian nucleic acid molecules listed in Tables 1 and 2 or encoding a protein listed in Table 1.

By “nucleic acid” is meant an oligomer or polymer of ribonucleic acid or deoxyribonucleic acid, or analog thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton, J. E. and Weller, D. D., U.S. Pat. No: 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (P. E. Nielsen et al. Science 199: 254, 1997). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃; OCF₃; O—, S—, or N-alkyl; O—, S—, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines.

By “nucleic acid molecule required for cell survival in the absence of Rb” is meant a nucleic acid molecule, or an ortholog thereof, whose inactivation (e.g., by RNAi) results in sterility, embryonic lethality, larval lethality, or larval growth arrest prior to adulthood in a nematode or cell death in a mammalian cell lacking functional Rb or a functional Rb pathway. Inactivation of such a gene in a mammalian cell lacking Rb results in cell death in at least 1%, 3%, 5%, 10%, 20%, 30%, 40%, 50%, 60%, or even in 70%, 80%, 90%, 95%, or 99% of the cells contacted with an inhibitory nucleic acid.

By “ortholog” is meant any polypeptide or nucleic acid molecule of an organism that is highly related to a reference protein or nucleic acid sequence from another organism. The degree of relatedness may be expressed as the probability that a reference protein would identify a sequence, for example, in a blast search. The probability that a reference sequence would identify a random sequence as an ortholog is extremely low, less than e⁻¹⁰, e⁻²⁰, e⁻³⁰, e⁻⁴⁰, e⁻⁵⁰, e⁻⁷⁵, e⁻¹⁰⁰. The skilled artisan understands that an ortholog is likely to be functionally related to the reference protein or nucleic acid sequence. In other words, the ortholog and its reference molecule would be expected to fulfill similar, if not equivalent, functional roles in their respective organisms.

It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs may be identified using methods provided herein. The functional role of an ortholog may be assayed using methods well known to the skilled artisan, and described herein. For example, function might be assayed in vivo or in vitro using a biochemical, immunological, or enzymatic assay; transformation rescue, or in a nematode bioassay for the effect of gene inactivation on nematode phenotype (e.g., fertility or vulval phenotype), as described herein. Alternatively, bioassays may be carried out in tissue culture; function may also be assayed by gene inactivation (e.g., by RNAi, siRNA, or gene knockout), or gene over-expression, as well as by other methods.

By “polypeptide” is meant any chain of amino acids, or analogs thereof, regardless of length or post-translational modification (for example, glycosylation or phosphorylation).

By “polypeptide required for cell survival in the absence of Rb” is meant a protein that is required for cellular viability in a nematode or in a mammalian cell that lacks functional Rb or a functional Rb pathway.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “purified antibody” is meant an antibody that is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “Rb biological activity” is meant the inhibitory effect of Rb or of a component of an Rb pathway on apoptosis or cellular proliferation or its opposition to oncogenic pathways (e.g., Rb activity opposes the Ras oncogenic pathway). Other Rb biological activities (e.g., promoting cellular senescence or differentiation, suppressing tumorigenesis) and methods of assaying Rb biological activities are known in the art and are described by, for example, Duanduan et al., J. Biol. Chem., 278:19358-19366, 2003 and by Harbour et al., Genes and Dev. 14:2393-2409, 2000). In addition, worm-based in vivo assays for Rb biological activity (e.g., Rb's effect on the C. elegans synMuv phenotype and embryonic development) are also known in the art and are described by, for example, Lu et al., Cell 95:981-91, 1998; Ceol et al., Mol. Cell 7:461-73, 2001; and Page et al., Mol. Cell 7:451-60, 2001.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “siRNA” is meant a double stranded RNA. Optimally, an siRNA is 18, 19, 20, 21, 22, 23 or 24 nucleotides in length and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

“Therapeutic compound” means a substance that has the potential of affecting the function of an organism. Such a compound may be, for example, a naturally occurring, semi-synthetic, or synthetic agent. For example, the test compound may be a drug that targets a specific function of an organism. A test compound may also be an antibiotic or a nutrient. A therapeutic compound may decrease, suppress, attenuate, diminish, arrest, or stabilize the development or progression of disease, disorder, or infection in a eukaryotic host organism.

By “transformed cell” is meant a cell into which (or into an ancestor of which) has been introduced, by means of recombinant DNA techniques, a polynucleotide molecule encoding (as used herein) a polypeptide of the invention.

By “transgene” is meant any piece of DNA that is inserted by artifice into a cell and becomes part of the genome of the organism that develops from that cell or, in the case of a nematode transgene, becomes part of a heritable extrachromosomal array. Such a transgene may include a gene which is partly or entirely heterologous (i.e., foreign) to the transgenic organism, or may represent a gene homologous to an endogenous gene of the organism.

By “transgenic” is meant any cell that includes a DNA sequence which is inserted by artifice into a cell and becomes part of the genome of the organism which develops from that cell or part of a heritable extrachromasomal array. As used herein, the transgenic organisms are generally transgenic invertebrates, such as C. elegans, or vertebrates, such as, zebrafish, mice, and rats, and the DNA (transgene) is inserted by artifice into the nuclear genome or into a heritable extrachromasomal array.

The invention provides a number of targets that are useful for the development of highly specific drugs to treat neoplasia or a disorder characterized by the deregulation of the cell cycle (e.g., a hyperproliferative disorder). In addition, the methods of the invention provide a facile means to identify therapies that are safe for use in eukaryotic host organisms (i.e., compounds that do not adversely affect the normal development, physiology, or fertility of the organism). In addition, the methods of the invention provide a route for analyzing virtually any number of compounds for effects on cell survival in the absence of Rb with high-volume throughput, high sensitivity, and low complexity. The methods are also relatively inexpensive to perform and enable the analysis of small quantities of active substances found in either purified or crude extract form.

Other features and advantages of the invention will be apparent from the detailed description, and from the claims.

### BRIEF DESCRIPTION OF THE DRAWINGS

FIG. 1 is a schematic diagram depicting the Rb pathway and other regulators of Rb function. The Rb pathway includes p16INK4, cyclin D, CDK4, Rb, E2F and DP. C. elegans Rb pathway homologs include cyd-1Cyclin D, cdk-4 CDK4, lin 35 Rb, efl-1 E2F, and dpl-1 DP. In addition, cki-1 is the worm homolog of p21^{CIP}, which is not part of the Rb pathway.

FIG. 2 is a schematic diagram depicting the effect of Rb on E2F heterodimers. Rb binding to activating E2F heterodimers turns them from activators of transcription to repressors. Rb binding to inhibitory E2F heterodimers allows them to translocate to the nucleus where they repress transcription. E2F heterodimers regulate transcription of genes responsible for cell cycle progression.

FIG. 3A is a schematic diagram depicting the effect of loss of Rb and p53 on a cell. When Rb is inactive, unrestrained E2F activity leads to activation of p53. p53 activation results in cell cycle arrest or programmed cell death; the factor that decides between these two options is not known.

FIG. 3B is a schematic diagram showing that when both Rb and p53 are inactivated, unrestrained E2F activity results in unregulated cell division.

FIG. 4 is a schematic diagram comparing the worm vulval induction pathway with the mammalian cell cycle regulation. In worms, the synMuvA and synMuv B pathways work together to oppose the Ras-MAP kinase pathway, which positively regulates vulval induction. lin-35 Rb, efl-1 E2F, dpl-1 DP, and hda-1 HDAC are synMuv B genes. In mammals, the Rb pathway blocks cell cycle progression induced by Ras.

### DETAILED DESCRIPTION

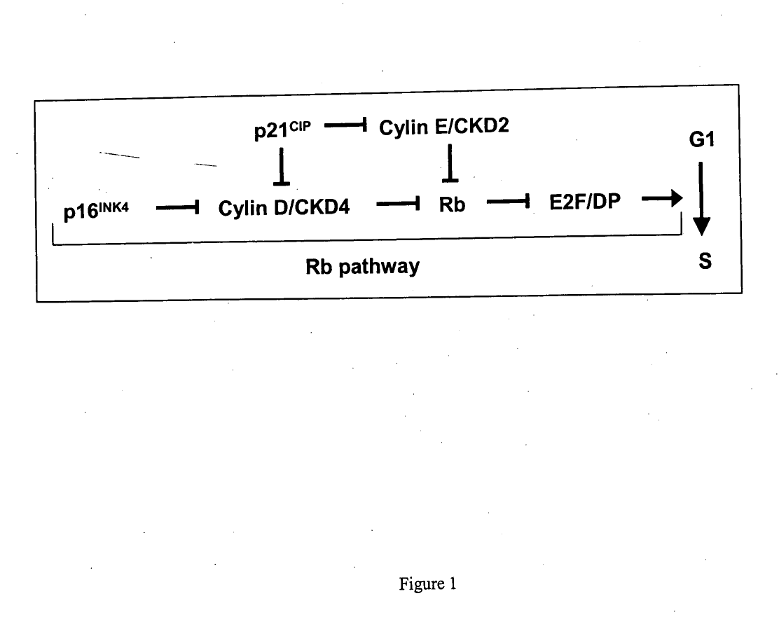

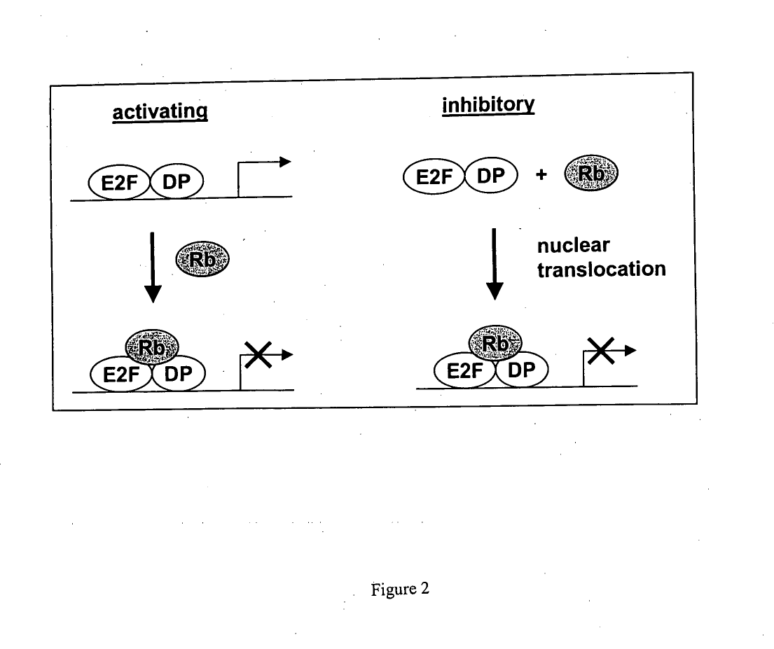

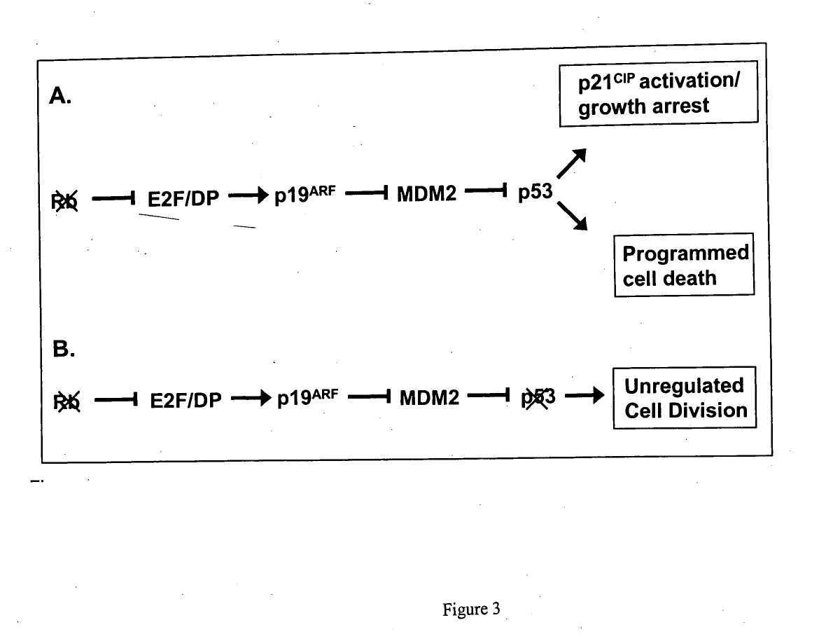

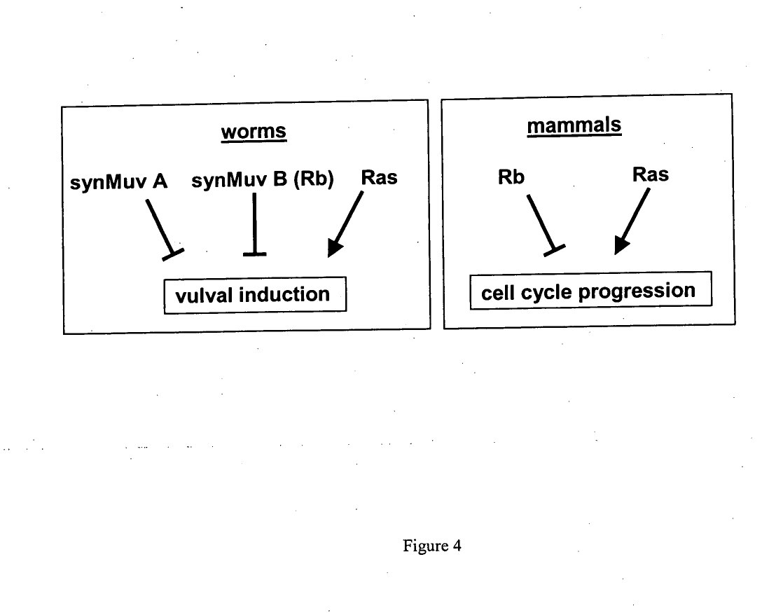

As described below, the present invention features nucleic acid and amino acid sequences required for cell survival in the absence of Rb. These sequences may be used in screening methods for identifying therapeutics for neoplasia treatment. The Rb pathway (FIG. 1) regulates entry of the cell into S phase from G1. When Rb is active, it blocks progression of the cell cycle by modulating the function of E2F heterodimeric transcription factors. The E2F family of transcription factors forms heterodimers (FIG. 2) with one of two DP proteins. Different combinations of E2F heterodimers either activate or repress transcription of genes that induce cell cycle progression. When Rb binds to activating heterodimers, it converts them from transcriptional activators to transcriptional repressors (Flemington et al., Proc Natl Acad Sci USA 90: 6914-8, 1993; Helin et al., Cell 70:337-50, 1992). Inhibitory heterodimers require Rb binding to bring them to the nucleus, thus allowing them to repress transcription (Muller et al. Mol Cell Biol 17, 5508-20, 1997); Verona et al. Mol Cell Biol 17:7268-82, 1997) (FIG. 3). The effects of losing Rb are shown in FIG. 3. When Rb is lost or inactivated, E2F activity is unrestrained, leading to activation of p53, which results in either cell cycle arrest or programmed cell death (FIG. 4). When both Rb and p53 are inactivated, unrestrained E2F activity results in unregulated cell division, often resulting in the development of cancer.

Rb pathway inactivation is a characteristic of cancer cells that can be used to search for cancer therapy targets that specifically target cells having an inactive Rb pathway for cell death, while leaving normal cells with an intact Rb pathway unharmed. The observation that both cancer cells and C. elegans can survive without an intact Rb pathway, suggests that cancer cells and C. elegans contain parallel pathways capable of substituting for the Rb pathway. As described in more detail below, we have used C. elegans to identify proteins that are specifically required for cell survival in cells lacking Rb. The inactivation of such proteins results in cell death in cells lacking Rb, but such inactivation does not harm cells with a functioning Rb pathway.

Proteins required for cell survival in the absence of Rb were identified by targeting the genes encoding them using RNA interference (RNAi) in mutant C. elegans lacking Rb, and in C. elegans having intact Rb. Proteins required for cell survival in the absence of Rb, but not required for cell survival in cells having an intact Rb pathway, likely act on processes in which Rb has a role, and hence, are necessary in the absence of Rb. Such proteins are promising targets for novel anti-cancer therapies. Drugs targeting such proteins would be expected to specifically effect cancer cells, thus reducing adverse side-effects and would be unlikely to be subject to chemoresistance.

Functional Genomic Screen using RNAi Library Feeding

A library of bacterial clones that express dsRNAs corresponding to almost every gene in the C. elegans genome has been generated. This library comprises 16,757 clones that target for inactivation approximately 86% of C. elegans genes. The results of a screen of N2 (wild-type) worms on the clones from Chromosome I were published in 2000 by Fraser et al. (Nature 408:325-30, 2000). We have screened lin-35(n745) worms against 2439 of the 2445 clones in the Chromosome I library.

We have followed the method outlined by Fraser et al. (Nature 408:325-30, 2000) for feeding RNAi. Specifically, lin-35(n745) L4 worms were plated on IPTG-containing plates seeded with bacteria expressing a particular dsRNA corresponding to a C. elegans gene. The animals were plated at 15° C. to slow the growth of the worms to allow for convenient screening. 72 hours after the original plating, three worms were transferred to three new plates seeded with dsRNA-expressing bacteria and the progeny was scored for sterility, embryonic lethality, larval lethality, larval growth arrest, or growth delay phenotypes on days 2, 5 and 10 after plating. The results were compared to the published results of the RNAi screen of Chromosome I in wild-type worms. All clones that gave a different phenotype in lin-35(n745) worms from the published results in wild-type worms were re-tested against both wild-type and lin-35(n745) strains. RNAi of genes that caused severe phenotypes in wild-type worms acted as internal controls, since one would expect to see a severe phenotype in lin-35(n745) worms as well. In fact, for chromosome I, all dsRNAs that caused severe phenotypes in wild-type worms, caused corresponding phenotypes in lin-35(n745) worms (see below).

Of the 2439 dsRNAs screened, 212 caused the same phenotypes in wild-type (N2) and lin-35(n745) worms. In a first screening 145 dsRNAs caused a different phenotype in lin-35(n745) worms than in N2 worms. On retesting against both strains, 105 dsRNAs still caused a phenotype in lin-35(n745) that was not observed in N2 worms. Since each experiment was done in triplicate, the effects of re-tested dsRNAs were examined at least six times.

dsRNAs were categorized into severe or mild classes based on the following criteria: severe phenotypes were any in which the dsRNAs caused sterility, embryonic lethality, larval lethality, or larval growth arrest prior to L3 in lin-35(n745) worms, but where no abnormalities or only minimal growth delay was observed in N2 worms. Mild phenotypes were those in which the dsRNAs caused lin-35(n745) phenotypes that were stronger than the N2 phenotypes, but where these phenotypes were not severe phenotypes. 73 dsRNAs that caused severe phenotypes and 32 dsRNAs that caused mild phenotypes were identified. Target genes corresponding to these dsRNAs are listed in Table 1. This list identifies C. elegans nucleic acids and their mammalian orthologs that are required in the absence of Rb. The target genes are identified by C. elegans cosmid name and open reading frame number. The human orthologs of the C. elegans genes are identified by gene name and by the GenBank accession number of the encoded protein. The sequences of the identified genes are available at http://www.ncbi.nlm.nih.gov/entrez/query.fcgi?CMD=Search&DB=nucleotide.

TABLE 1
C. elegans Genes and Human Orthologs

| | Human Orthologs (% identity) | |
|---|---|---|
| Gene | Gene Bank Accession numbers | Function/Identity |
| Replication | | |
| C43E11.10 | CDC6 (28%): NP_001245 | involved in cell cycle regulation of replication |
| | ORC1L (28%): NP_004144 | initiation |
| | ORC4L (24%): NP_002543 | |
| M04F3.1 | RPA2 (22%), 41% similarity: | subunit of ssDNA-binding protein complex |
| | NP_002937 | replication protein A; roles in DNA replication, |
| | | repair, and recombination |
| F28B3.7 | SMC1L1 (42%): Q14683, | SMC family - putative role in chromosome |
| | SMC1L2 (38%): NP_683515 | segregation |
| RNA | | |
| metabolism | | |
| C55B7.8 | DBR1 (45%): NP_057300 | lariat RNA debranching enzyme involved in turnover |
| | FLJ10998: NP_060764 | of excised spliceosomal introns |
| T21G5.3 | DDX4 (35%): AAH47455 | DEAD box RNA helicase glh-1 in P granules |
| | DDX3 (38%): AAC34298 | |
| | DBY (39%): AAC51832 | |
| F22D6.5 | PRP4 (41%): NP_789770 | PRP4 nuclear S/T kinase with role in regulation of |
| | HIPK3 (31%): NP_005725 | pre-mRNA splicing |
| | HIPK2 (31%): AAG41236 | |
| R05D11.7 | RY1 (52%): NP_006848 | putative nucleic acid binding protein |
| C25A1.3 | RNMT (40%): BAA23694 | mRNA cap methyltransferase |
| C44E4.4 | SSB (34%); 33% similarity to | moderate similarity to Sjogren Syndrome Antigen B; |
| | human La: P05455 | RNA binding motif |
| C06A5.5 | | SR splicing factors; C. elegans F44F4.2 (64%); TC8.1 |
| | | (86%); F14D2.10 (81%). SFRS4 (39%) over 64 a.a.s |
| C37A2.4 | | RNA-binding protein like T-STAR, GLD-1 |
| T25G3.3 | LOC51068 (51%): NP_057022 | necessary for stable 60s ribosomal subunit formation; |
| | | yeast nonsense mRNA mediated decay protein like |
| T05E8.3 | DDX33 (36%): NP_064547 | putative ortholog of DDX33; putative DEAH box |
| | DDX8 (35%): NP_004932 | RNA helicase |
| | DDX38 (32%): NP_054722 | |
| D1081.8 | CDC5L (47%): NP_001244 | transcriptional activator and non-snRNA spliceosome |
| | | subunit involved in pre-mRNA splicing, 2 Myb-like |
| | | DNA-binding domains |
| B0511.6 | DDX18 (63%): NP_006764 | putative ortholog of DDX18; Member of DEAD or |
| | | DEAH box ATP-dependent RNA helicase family |
| Y105E8C.e | | snRNP polypeptide B |
| Transcription | | |
| Y40B1B.6 | KIAA0601 (28%): AAH48134, | flavin containing amino oxidase; member of HDAC1 |
| | C20ORF16 (24%): NP_787033 | complex |
| ZC123.3 | ATBF1 (23%); 33% similarity: | 10 Zn finger and 3 homeobox domains; AT binding |
| | NP_008816 | motif; transcriptional repressor in neural and muscle |
| | | differentiation |
| H06O01.3 | SEC14L2 (30%): NP_036561 | CRAL-TRIO domain binds alpha-tocopherol, |
| | | translocates from cytosol to nucleus to activate |
| | | transcription |
| K02B12.1 | | ceh-6, homeobox transcription factor |
| Y47H9C.7 | RNAP3 (22%): NP_055054 | RNA pol III subunit |
| Y65B4A_182.c | | transcription activator |
| C48E7.2 | RPC62 (24%): AAM12033 | RNA pol III subunit that functions in specific |
| | | transcription initiation |
| F14B4.3 | RPO1-2 (55%): NP_061887 | RNA pol beta subunit family member |
| | POLR2B (29%): NP_000929 | |
| | FLJ10388 (40%): NP_060552 | |
| W04A8.7 | TAF1 (36%): NP_004597 | putative ortholog of TBP assoc factor. Assoc |
| | | W/RNAP II. 2 bromodomains |
| Translation | | |
| R12E2.12 | | Possible mitochondrial small ribosomal subunit |
| | | protein |
| Y106G6H.3 | RPL30 (72%): P04645 | 60S ribosomal subunit, binds DNA, member of the |
| | | ribosomal L7Ae, L30e, S12e or Gadd45 family with |
| | | ribosomal and growth reg functions |
| C43E11.9 | HSPC031 (61%): AAD40195 | Putative ortholog of nucleolar protein yeast Nip7p req |
| | | for 60S biogenesis |
| T25G3.3 | CGI-07, LOC51068 (51%): | necessary for stable 60s ribosomal subunit formation |
| | NP_057022 | |
| C03D6.8 | C15ORF15 (55%): NP_057388 | 60S ribosomal protein L30 isolog (L24e family |
| | | member) |
| T23D8.4 | EIF3S8 (43%): NP_003743 | putative ortholog of translation initiation factor 3 |
| Miscellaneous | | |
| F46F11.4 | UBL5 (81%): NP_077268 | strong similarity to Ubiquitin family |
| B0207.6 | FLJ10349 (43%): NP_060536 | strong similarity to conserved hypothetical ATP |
| | | binding protein family |
| C55B7.6 | SLC26A2 (22%): NP_000103 | wk similarity to sulfate transporters |
| | SLC26A8b (20%): NP_619732 | |
| | SLC26A8a: NP_443193 | |
| | SLC26A1a (19%): NP_071325 | |
| | SLC26A1b: NP_602297, | |
| | SLC26A1c: NP_602298 | |
| M05B5.2 | | C. elegans F28B4.4 (49%); myo-2 B |
| F07A5.1 | | Drosophila INX2 (24%) over 333 a.a.; opu-14; unc-7 |
| | | like, ogre family, inx-14, innexin (a and b forms). |
| F27D4.1 | ETFA (65%): AAH15526 | electron-transfer-flavoprotein, alpha polypeptide |
| | | (glutaric aciduria II)) |
| F45H11.2 | UBA52 (62%): NP_003324 | ned-8: ubiquitin with roles in seam cell diff, |
| | RPS27A (62%): UQHUR7 | cytoskeletal reg of pronuc mig, cytokinesis, meiosis to |
| | UBB (62%): AAH15127 | mitosis |
| R06C1.3 | WASF1 (29%): NP_003922 | WASP family Verprolin-homologous protein |
| W09C5.8 | COX4I1 (29%): AAH21236, | cytochrome oxidase subunit 4 |
| | COX4I2 (30%): NP_115998 | |
| Y54E5A.4 | MUC1 (30%)¹: NP_877418 | multiple GLFG repeats (found in many nucleoporins) |
| R06A10.2 | GNAS (65%): AAP88907 | gsa-1 Gs alpha subunit similar to goa-1 |
| | GNAL (64%): NP_002062 | |
| M01B12.3 | ARPCS (42%): NP_005708, | ARP2-3 complex subunit 5 |
| | MGC3038 (39%): NP_112240 | |
| R12E2.10 | | PTPase high sim to C. elegans T21E3.1 involved in |
| | | eggshell formation. PTPN9 (32%) over 273 aa (753 |
| | | aa). |
| F55A12.7 | AP1M1 (72%): Q9BXS5 | clathrin associated protein |
| C01H6.7 | BRD7 (28%): NP_037395, | may interact with PDZ domain from tyrosine |
| | FLJ13441 (48%): NP_076413, | phosphatase |
| | BRD1 (27%): NP_055392 | |
| F30A10.6 | SACM1L (47%): AAH16559 | high similarity to suppressor of actin, integral |
| | SAC2 (36%): AAH32108 | membrane lipid phosphatase, acts on PI3P, PI4P, |
| | SYNJ1 (33%): NP_003886 | PI4, 5BP |
| T23D8.1 | FZD1 (40%): AAH51271 | |
| | FZD7 (39%): O75084 | |
| | FZD2 (38%): AAH52266 | |
| Y52B11A.9 | KIN (47%): NP_036443 | sim to region of RecA; Zn finger C2H2; ribosomal |
| | | prot L6 signature 1 |
| T04D3.3 | PDE1A (49%): NP_005010 | high similarity to Ca/calmodulin dependent cyclic |
| | PDE1C (48%): NP_005011 | nucleotide phosphodiesterase |
| | PDE1B (47%): NP_000915 | |
| Y40B1A.4 | | 3 Zn finger C2H2 domains |
| C01A2.3 | OXAOXA1L (27%)²: | inner mito membrane protein required for assembly of |
| | NP_0050061L | F1F0-ATP synthase and cytochrome oxidase and |
| | | export of cox2p |
| Y87G2A.s | VPS28 (46%): AAH50713 | homology to S. cereviseae vacuolar trafficking protein |
| Y6B3A.1 | ARFGEF2 (42%): NP_006411 | non-clathrin coated vesicle protein GEF family |
| Y54E10_156.a | | adaptin complex subunit |
| K07A12.2 | | 14 leucine rich repeats, LIB (29%) over 428 aa (961 |
| | | aa) |
| C43H8.3 | | Protein containing an extracellular laminin G domain |
| C32E8.5 | | FHA domain |
| T03F1.8 | GUK1: AAH09914 | kin-10 reg subunit of CKII beta (guanylate kinase 1) |
| F57C9.4 | | C. elegans Y48A6B.8 (41%) over 320 residues; 3 |
| | | C2H2 Zn finger domains |
| F28B3.1 | KIAA0934 (52%): Q9Y2E4 | AMP binding protein enzyme family |
| T19B4.4 | MCJ (56%): NP_037370 | contains DnaJ domain, chaperone interacting domain. |
| C10G11.5 | FLJ10782 (40%): NP_060686 | putative pantothenate kinase involved in coenzyme A |
| | | biosynthesis |
| T21G5.4 | | strong sim to C. elegans C25G4.6, required for |
| | | spermatogen, 2 PDZ, DHR, or GLGF domains, found |
| | | in signaling proteins |
| C30F12.4 | | MSF is septin like GTP binding protein fused to MLL |
| | | in AML and maps to region deleted in ovarian tumors |
| F27D4.5 | BCKDHB (66%): NP_000047 | branched chain alpha keto acid dehydrogenase E1 |
| | | beta subunit assoc with maple syrup urine disease |
| C09H6.1 | ZNF208 (22%): NP_009084 | 17 C2H2 Zn finger domains |
| C34B7.3 | CYP2A13 (33%): AAG35775, | cytochrome P450 cyp2 family similarity |
| | CYP2A7 (34%): NP_000755, | |
| | CYP2A6 (33%): P11509 | |
| F30A10.10 | NP_115612 | putative ubiquitin-specific protease |
| F26E4.6 | NP_001858 | Member of cytochrome c oxidase subunit VIIc family, |
| | | part of terminal oxidase in mitochondrial electron |
| | | transport chain |
| Unknown | | |
| function | | |
| F27C1.6 | KIAA0266 (27%): NP_067677 | unknown function |
| C55B7.9 | FLJ20045 (32%): NP_060108 | unknown function |
| C36B1.8 | | unknown function |
| ZK858.2 | | unknown function |
| ZK39.6 | | unknown function |
| K08C9.1 | | unknown function |
| Y34D9A_151.a | | unknown function |
| Y54E10_155.c | | unknown function |
| Y54E10_155.e | | unknown function |
| R119.6 | | unknown function |
| C10H11.10 | | C. elegans paralog M04F3.3 (98%); unknown |
| | | function |
| F48C1.4 | | unknown function |
| F55A12.8 | FLJ10774 (54%): NP_078938 | unknown function |
| ZC308.2 | | unknown function |
| ZK265.6 | HSPC111 (30%): NP_057475 | unknown function |
| F30A10.9 | CGI-35, LOC51077 (58%): | unknown function |
| | NP_057046 | |
| T23D8.3 | | Drosophila LD21529 (28%); unknown function |
| Y95D11A.a | | unknown function |
| Y87G2A.e | | unknown function |
| Y71A12B.b | | unknown function |
| Y48G8A_3671.a | | unknown function |
| Y65B4A_174.b | | unknown function |
| ZC123.2 | | unknown function |
| R12E2.2 | | Drosophila CG17466 (23%) over 637 a.a. unknown |
| | | function, C1ORF9 (41%) over 226 (800 a.a. prot) |
| T19B4.5 | | unknown function |
| C26C6.5 | | unknown function |
| F02E9.3 | F36A2.12 (27%): XP_293124 | unknown function |
| F39H2.3 | | Drosophila CG8043 (30%) unkown function |
| ZK858.7 | LOC51605 (25%) | unknown function |
| C03D6.1 | | C. elegans CO4F12.1 (25%), unknown function |
| Y39G10A_246.I | | unknown function |
| Y39G10A_246.j | | unknown function |
| Y47G6A_247.h | | unknown function |
| Y48G1A_54.d | | unknown function |
| Y54E10B_159.e | | unknown function |

¹moderate similarity over 206 amino acids (538 aa)
²27% over 324 amino acids

Of the 105 dsRNAs that had synthetic phenotypes with lin-35(n745) worms, 50 have human homologs, defined as having at least 25% identity over 75% of the genes. C. elegans genes with identified human orthologs are shown in Table 2. Additional orthologs may be identified as described herein. This list identifies the target genes by C. elegans cosmid name and open reading frame number. The human orthologs of the C. elegans genes are identified by gene name. These human genes are likely required for mammalian cell survival in cells lacking Rb. The sequences of the identified human genes are available at http://www.ncbi.nlm.nih.gov/entrez/query.

TABLE 2
Human Orthologs of C. elegans Genes Required in Cells Lacking Rb

| | Synthetic | Human gene | |
|---|---|---|---|
| Gene | phenotype | homologs (% identity) | Function/identity |
| Replication | | | |
| C43E11.10 | severe | CDC6 (28%) | involved in cell cycle regulation of replication |
| | | ORC1L (28%) | initiation |
| | | ORC4L (24%) | |
| M04F3.1 | severe | RPA2 (22%)³ | subunit of ssDNA-binding protein complex |
| | | | replication protein A; roles in DNA replication, |
| | | | repair, and recombination |
| F28B3.7 | severe | SMC1L1 (42%); | SMC family - putative role in chromosome |
| | | SMC1L2 (38%); | segregation |
| | | SMC4L1 (24%) | |
| RNA | | | |
| metabolism | | | |
| C55B7.8 | severe | DBR1 (45%) | lariat RNA debranching enzyme involved in |
| | | | turnover of excised spliceosomal introns |
| T21G5.3 | severe | DDX4 (35%) | DEAD box RNA helicase glh-1 in P granules |
| | | DDX3 (38%) | |
| | | DBY (39%) | |
| F22D6.5 | severe | PRP4 (41%) | PRP4 nuclear S/T kinase with role in regulation of |
| | | HIPK3 (31%) | pre-mRNA splicing |
| | | HIPK2 (31%) | |
| R05D11.7 | severe | RY1 (52%) | putative nucleic acid binding protein |
| C25A1.3 | severe | RNMT (40%) | mRNA cap methyltransferase |
| C44E4.4 | severe | SSB (34%)⁴ | moderate similarity to Sjogren Syndrome Antigen |
| | | | B; RNA binding motif |
| T05E8.3 | mild | DDX33 (36%) | putative ortholog of DDX33; putative DEAH box |
| | | | RNA helicase |
| D1081.8 | mild | CDC5L (47%) | transcription activator and non-snRNA |
| | | | spliceosome subunit involved in pre-mRNA |
| | | | splicing, 2 Myb-like DNA-binding domains |
| B0511.6 | mild | DDX18 (63%) | putative ortholog of DDX18; Member of DEAD or |
| | | | DEAH box ATP-dependent RNA helicase family |
| Transcription | | | |
| Y40B1B.6 | severe | KIAA0601 (28%)⁵ | flavin containing amino oxidase; member of |
| | | | HDAC1 complex |
| ZC123.3 | severe | ATBF1 (23%)⁶ | 10 Zn finger and 3 homeobox domains; AT |
| | | | binding motif; transcriptional repressor in neural |
| | | | and muscle differentiation |
| H06O01.3 | severe | SEC14L2 (30%) | CRAL-TRIO domain binds alpha-tocopherol, |
| | | | translocates from cytosol to nucleus to activate |
| | | | transcription |
| C48E7.2 | mild | RPC62 (24%) | RNA pol III subunit that functions in specific |
| | | | transcription initiation |
| F14B4.3 | mild | RPO1-2 (55%); | RNA pol beta subunit family member |
| | | POLR2B (29%); | |
| | | FLJ10388 (40%) | |
| W04A8.7 | mild | TAF1 (36%) | putative ortholog of TBP assoc factor. Assoc |
| | | | w/RNAP II. 2 bromodomains |
| Translation | | | |
| Y106G6H.3 | severe | RPL30 (72%) | 60S ribosomal subunit, binds DNA, member of |
| | | | the ribosomal L7Ae, L30e, S12e or Gadd45 |
| | | | family with ribosomal and growth regulatory |
| | | | functions |
| C43E11.9 | severe | HSPC031 (61%)⁷ | Putative ortholog of nucleolar protein yeast |
| | | | Nip7p req for 60S biogenesis |
| T25G3.3 | severe | LOC51068 (51%) | necessary for stable 60s ribosomal subunit |
| | | | formation |
| C03D6.8 | severe | C15ORF15 (55%) | 60S ribosomal protein L30 isolog (L24e family |
| | | | member) |
| T23D8.4 | severe | EIF3S8 (43%) | putative ortholog of translation initiation factor 3 |
| Miscellaneous | | | |
| F46F11.4 | severe | UBL5 (81%) | strong similarity to Ubiquitin family |
| B0207.6 | severe | FLJ10349 (43%) | strong similarity to conserved hypothetical ATP |
| | | | binding protein family |
| F27D4.1 | severe | ETFA (65%) | electron-transfer-flavoprotein, alpha polypeptide |
| | | | (glutaric aciduria II)) |
| F45H11.2 | severe | UBA52 (62%) | ned-8: ubiquitin with roles in seam cell diff, |
| | | RPS27A (62%) | cytoskeletal reg of pronuc mig, cytokinesis, |
| | | UBB (62%) | meiosis to mitosis |
| R06C1.3 | severe | WASF1 (29%) | WASP family Verprolin-homologous protein |
| W09C5.8 | severe | COX4I1 (29%); COX4I2 | cytochrome oxidase subunit 4 |
| | | (30%) | |
| R06A10.2 | severe | GNAS (65%) | gsa-1 Gs alpha subunit similar to goa-1 |
| | | GNAL (64%) | |
| M01B12.3 | severe | ARPC5 (42%); | ARP2-3 complex subunit 5 |
| | | MGC3038 (39%) | |
| F55A12.7 | severe | AP1M1 (72%) | clathrin associated protein |
| C01H6.7 | severe | BRD7 (28%)⁸ | may interact with PDZ domain from tyrosine |
| | | | phosphatase |
| F30A10.6 | severe | SACM1L (47%) | high similarity to suppressor of actin, integral |
| | | SAC2 (36%) | membrane lipid phosphatase, acts on PI3P, PI4P, |
| | | SYNJ1 (33%) | PI4, 5BP |
| T23D8.1 | severe | FZD1 (40%) | frizzled-like protein |
| | | FZD7 (39%) | |
| | | FZD2 (38%) | |
| T04D3.3 | severe | PDE1A (49%) | high similarity to Ca/calmodulin dependent |
| | | PDE1C (48%) | cyclic nucleotide phosphodiesterase |
| | | PDE1B (47%) | |
| C01A2.3 | severe | OXA1L (27%)⁹ | inner mito membrane protein required for |
| | | | assembly of F1F0-ATP synthase and cytochrome |
| | | | oxidase and export of cox2p |
| Y87G2A.s | severe | VPS28 (46%) | homology to S. cereviseae vacuolar trafficking |
| | | | protein |
| Y6B3A.1 | severe | ARFGEF2 (42%) | non-clathrin coated vesicle protein GEF family |
| | | BIG1 (41%) | |
| F28B3.1 | mild | KIAA0934 (52%) | AMP binding protein enzyme family |
| C10G11.5 | mild | FLJ10782 (40%) | putative pantothenate kinase involved in |
| | | | coenzyme A biosynthesis |
| F27D4.5 | mild | BCKDHB (66%) | branched chain alpha keto acid dehydrogenase E1 |
| | | | beta subunit assoc with maple syrup urine disease |
| C34B7.3 | mild | CYP2A13 (33%); | cytochrome P450 cyp2 family similarity |
| | | CYP2A7 (34%); | |
| | | CYP2A6 (33%) | |
| Unknown | | | |
| function | | | |
| F27C1.6 | severe | KIAA0266 (27%) | unknown function |
| C55B7.9 | severe | FLJ20045 (32%) | unknown function |
| F55A12.8 | severe | FLJ10774 (54%) | unknown function |
| ZK265.6 | severe | HSPC111 (30%) | unknown function |
| F30A10.9 | severe | LOC51077 (58%) | unknown function |
| F02E9.3 | mild | F36A2.12 (27%) | unknown function |
| ZK858.7 | mild | LOC51605 (25%) | unknown function |

³41% similarity
⁴33% similarity to human La
⁵46% similarity over 576 amino acids
⁶33% similarity
⁷80% similarity
⁸46% similarity
⁹over 324 amino acids

C. elegans and mammalian genes required in cells that lack Rb or a functional Rb pathway have been categorized according to function. 38 of the 73 dsRNAs that caused severe phenotypes have human homologs (52.1%); 12 of 32 dsRNAs that caused mild phenotypes have human homologs (37.5%). The list contains a number of promising candidates for genes in Rb-related or parallel pathways. Three dsRNAs (e.g., C43E11.10, M04F3.1, F28B3.7) correspond to genes involved in DNA replication or its regulation. Given that Rb is the restriction point switch responsible for determining whether the cell replicates its DNA in S phase, it is tempting to speculate that these genes (e.g., C43E11.10, M04F3.1, F28B3.7) encode downstream effectors of the Rb pathway or a parallel pathway. Another promising candidate, Y40B1B.6, is the homolog of a flavin containing amino oxidase that is a member of a HDAC1 complex. HDAC participates in a complex with Rb and E2F/DP responsible for transcriptional repression of many genes and hda-1 HDAC is a synMuv B gene.

71 dsRNAs that were reported to have effects on growth of N2 worms did not have any effects on lin-35(n 745) worms. When we repeated these experiments with lin-35(n745) and N2 worms we observed no effects in either strain. For eight of these dsRNAs (those in the first 10% of the library screened), the plasmids encoding them were isolated from the bacterial strains and sequenced. Two of these did not provide readable sequence and were considered to have incorrect inserts. The other six were used to make RNA in vitro for injection into N2 worms since RNAi by injection is more penetrant and reliable than RNAi by feeding. Of these six dsRNAs, one caused larval arrest and one caused a mild growth delay when injected. The others had no discernible effect on worm growth or morphology. Overall, the results of RNAi by injection did not differ significantly from the results of feeding RNAi, confirming the effectiveness of the feeding RNAi approach.

Studies of Other Genes in the Rb Pathway

One of the advantages of the C. elegans system is that mutants in genes known to interact with lin-35 Rb exist. dsRNAs that show synthetic phenotypes with lin-35 Rb will be tested in viable strains mutant for known lin-35 Rb interacting proteins, such as efl-1 E2F, dpl-1 DP, cyd-1 cyclin D, and hda-1 HDAC (Boxem et al. Development 128: 4349-592002; Ceol et al. Mol Cell 7:461-73, 2001; Lu et al., Cell 95, 981-91, 1998; Page et al., Mol Cell 7: 451-60, 2001). These studies may categorize identified genes into functional classes. For example, identified genes that are synthetically lethal with lin-35 Rb but not with efl-1 E2F are likely to represent new effectors that are independent of Rb, and act through E2F. Genes synthetically lethal with both lin-35 Rb and efl-1 E2F are likely to function in pathways parallel to the entire lin-35 Rb pathway. Indeed, severe mutants of dpl-1 and efl-1 are sterile (Ceol and Horvitz, supra), a more severe phenotype than is seen with a lin-35 null phenotype, implying that other gene products act through EFL-1 and DPL-1 in pathways parallel to the lin-35 Rb pathway. Because this screen is unbiased (i.e. any dsRNA synthetically lethal with lin-35(n745) is analyzed, regardless of whether it has a known function), it will likely identify pathways not previously identified in mammalian studies, which typically focus on proteins suspected of being directly involved in Rb pathway function. Given the large number of candidate genes already identified in approximately one sixth of the genome, categorizing these RNAs into functional classes is extremely important. The screening of dsRNAs with these strains is carried out as described above for lin-35(n745) worms.

Generation of Deletion Mutants

Mutant worms harboring a deletion in a gene of interest (e.g., a gene required for nematode survival in the absence of Rb) are made using a C. elegans deletion library and high throughput robotic screening methods to identify deletions. Briefly, worms are mutagenized to create deletions. Isolated genomic DNA from pools of mutagenized worms is then screened by PCR using nested primers surrounding a genetic region of interest. The PCR product amplified from a region having a deletion is smaller than a PCR product amplified from a wild-type worm not having a deletion. Once a pool of worms harboring a deletion in a gene of interest is identified, the worms are thawed and plated singly. Nested PCR is then carried out on genomic DNA from some of the progeny of each thawed worm. Worms having a deletion in the gene of interest are identified by the smaller size of the PCR product produced relative to the PCR product obtained from a wild-type worm. Identifying candidate genes that have synthetic defects when deleted (as opposed to having effects on their own when deleted), allows the identification of proteins that function in parallel pathways to Rb. The deletion approach is particularly advantageous, because deletions affect all tissues equally, while some tissues, such as neurons, are less sensitive to RNAi. Thus, deletion analysis may identify tissue-specific synthetic phenotypes not identified by RNAi.

Mosaic Analysis

Alternatively, animals mosaic for lin-35 Rb inactivation (Fay et al., Genes Dev 16:503-17, 2002) in specific tissues are generated using standard methods. Using mosaics, one can assess the differential effects of targeting candidate genes for RNAi in tissues that do or do not express lin-35 Rb. This approach allows the synthetic effects to be evaluated in different tissues and at different stages of development depending on which cells are analyzed and when during development the lin-35 Rb gene is lost. Therefore, one may uncover unforeseen tissue-specific or developmental-specific roles for lin-35 Rb or the candidate genes.

Mosaic worms are typically made by constructing a strain having a loss of function in a gene of interest (e.g. lin-35(n745)) and a second loss of function in at least one marker gene whose expression is required in a specific cell lineage. The double mutant strain is transformed with an extrachromosomal array containing functional copies of both genes (Herman et al. Methods Cell Biol 48:123-46, 1995; Herman et al. Nature 348: 169-71, 1990; Miller et al., Genetics 143:1181-91,1996). The array is randomly lost in a subset of mitoses. Cells in a lineage that has lost the array display the phenotype of the loss-of-function marker gene. Cells that lose the marker gene have also lost the functional copy of the gene of interest, and therefore, can be assessed for the effect of the loss of that gene. For example, a loss-of-function mutation in ncl-1 (e1942) results in large nucleoli that are visible by differential interference contrast (DIC) microscopy (Miller et al., Genetics 143:1181-91,1996). Worms having mutations in both lin-35 and ncl-1, lin-35(n745); ncl-1 (e1942), worms containing an array with functional lin-35 Rb and ncl-1 can be grown on bacteria expressing dsRNA from a candidate gene synthetically lethal with lin-35(n745). If no worms are found with tissues containing large nucleoli, it is likely that cells that lost the array containing functional lin-35 Rb were killed by the effect of the synthetically lethal dsRNA. Hence, the synthetic effect is cell autonomous. Alternatively, if worms are observed to have cells containing large nucleoli all derived from the same lineage, then that lineage can survive without the array and, the synthetic effects of the candidate RNAi with lin-35 Rb are non-cell autonomous. Other tissue-specific markers genes, such as dpy-17 and unc-36 are also useful in such methods (Thomas et al. Development 126: 3449-59, 1999).

Tissue-Specific Screens for Cell-Autonomous Synthetic Lethality

Tissue-specific assays for synthetic lethality allow us to analyze the effects of disrupting gene function in a single tissue. lin-35 Rb mutant nematodes are less healthy than wild-type animals (decreased brood size and rare sterile animals), thus it is possible that some of the synthetic phenotypes observed in lin-35 Rb mutant nematodes, but not in wild-type nematodes are caused by the non-specific additive effects of two harmful mutations. For example, RNAi of a particular gene may affect one cell type while the lin-35 Rb mutation affects another, but the two defects in a single animal may result in a severely affected animals. For example, a lin-35 Rb mutation may decrease brood size via its effect on the cell cycle or on the germ-line itself, resulting in abortive germ-line mitoses. A dsRNA may have an effect on the somatic gonad and also decrease brood size. Together, the two effects may result in sterile animals, although they effect different cells and, at a molecular level, are essentially unrelated. Alternatively, a dsRNA may decrease the ability of an animal to feed causing malnutrition, which decreases its brood size or result in a larger percentage of dead eggs. When the effects of such a dsRNA are combined with the already decreased brood size of a lin-35 Rb animal, synthetic sterility or even synthetic embryonic lethality may occur. To identify redundant pathways within single cells, i.e. parallel pathways required for survival in cells such as neoplasia, it may be desirable to distinguish between dsRNAs that cause cell-autonomous synthetic lethality and non-cell-autonomous synthetic lethality (i.e. the synthetic effects of two mutations occur within a single cell rather than between cells). To address this issue, three tissue-specific assays for synthetic lethality are available.

Tissue-Specific Foldback lin-35 Rb RNAi

Tissue-specific depletion of lin-35 Rb can be generated by expressing an RNA with tandem inverted repeats of the lin-35 Rb gene from a tissue-specific promoter. This RNA should fold back on itself to create a dsRNA that targets lin-35 Rb transcripts. These foldback RNAs deplete endogenous mRNAs in a sequence-specific fashion. Candidate genes are then targeted for RNAi using this strain to look for tissue-specific cell death.

The efficacy of the foldback construct can be evaluated by growing the foldback construct expressing strain on bacteria that produce a dsRNA targeting a synMuv A gene. If the foldback construct is effective, the worms will display a Muv phenotype. The lin-35 Rb foldback construct will also be co-injected with gfp expressed from the same tissue-specific promoter to mark the tissue.

Tissue-Specific rde-1 Expression

Tissue-specific RNAi can be accomplished using worms mutant for the gene rde-1. rde-1 encodes a gene that is part of a complex responsible for processing dsRNAs into the form required for RNAi (Tabara et al., Cell 99:123-32, 1999). rde-1(ne219) worms are insensitive to RNAi. rde-1 (ne219) worms expressing wild-type rde-1 under the control of a tissue-specific promoter should only be susceptible to RNAi in tissues expressing wild-type rde-1. gfp can be used as a co-injection marker. gfp expressed from the same tissue-specific promoter as rde-1, or from other desirable promoters, can be co-injected with the rde-1 containing construct. Worms fed dsRNA corresponding to gfp are expected to have a decreased or absent GFP signal in the rde-1 expressing tissues, but a normal GFP signal in the other tissues.

Tissue Specific Promoters

Desirably, promoters for the tissue-specific assays described above are active in a tissue that is not necessary for the viability of a nematode and that is easily analyzed for abnormalities. Ideally, the promoter is active during multiple rounds of cell divisions, since lin-35 Rb is known to act as a cell cycle regulator and, therefore, its absence is expected to have a more significant effect on dividing cells. In one example, lin-35 Rb is expressed under the control of the lin-31 promoter (Miller et al., Genetics 143:1181-91,1996; Tan et al., Cell 93:569-80, 1998). The lin-31 gene encodes a transcription factor that is expressed only in the C. elegans vulva and vulval precursors. The lin-31 promoter is active prior to and during the cell divisions that create the vulva. This allows the effects of tissue specific RNAi to be evaluated in the developing vulva and provides the following advantages.

The vulva is not required for viability. A number of vulvaless (Vul) mutants exist, such as let-60 Ras loss of function mutants, as well as mutants with morphologically abnormal vulvae (Herman et al., Proc Natl Acad Sci U S A 96:968-73, 1999; Stemberg et al., Trends Genet 14: 466-72, 1998). Vul animals typically produce progeny that hatch inside the mother and devour her, producing a ‘bag of worms’, an easily observable phenotype. More subtle phenotypes are also easily detected, since the vulva is a large structure on the outside of the animal that goes through stereotyped changes that occur at the beginning of the third larval stage and continue through adulthood. Thus, promoters directing vulva-specific expression are useful in the methods of the invention.

In one example, GFP is expressed under the control of the lin-31 promoter in the above-described assays. Altered (e.g., decreased or absent) GFP-expression is indicative of tissue damage even if the vulva looks grossly normal. Changes in GFP expression may be detectable earlier than morphological defects also, since the vulva does not develop until the third larval stage. Furthermore, because it is possible that some morphological defects will compromise the integrity of the worm hypodermis, leading to premature death during vulval development, the GFP assay allows the identification of defects during early stages before lethality occurs.

In another example, the hlh-8 gene is used as a tissue specific promoter. hlh-8 encodes the C. elegans homolog of Twist (CeTwist), a basic helix-loop-helix protein active in part of the M lineage, which gives rise to a small number of body wall muscles, the sex-specific muscles (eight muscles that are responsible for egg-laying), the enteric muscles (responsible for defecation) and two coelomocytes (Corsi et al., Development 127:2041-512000; Harfe et al., Genes Dev 12: 2623-35, 1998). Ablation of the entire M lineage does not affect survival of the animal (Sulston and White, Dev Biol 78:577-97, 1980). Due to the lack of sex-specific and enteric muscles, worms that do not contain these cells have two visible phenotypes: they are bloated with eggs (Egl) and they are constipated (which results in a visibly enlarged intestine).

In another example, gld-1 is used as a tissue specific promoter. gld-1 encodes a cytoplasmic protein found in the germline that is required for mitotic germline cells to undergo meiosis and become gametes. Absence of GLD-1 expressing cells results in a sterile phenotype (Ste); the animals contain no eggs (Jones et al., Dev Biol 180:165-83, 1996).

Mammalian Orthologs of C. elegans RNAi Clones

Mammalian orthologs of C. elegans RNAi clones identified in Tables 1 and 2 are likely to be required in neoplastic mammalian cells that lack Rb. Such genes are particularly promising therapeutic targets for the treatment of neoplasia or diseases characterized by inappropriate cell-cycle regulation, since drugs that inactivate such genes are expected to specifically effect neoplastic cells and are unlikely to interfere with the function of normal cells. Such genes are particularly promising therapeutic targets for the treatment of neoplasia, since drugs that inactivate them are less likely than current therapies to cause adverse side-effects since they should not effect non-neoplastic tissues.

Identification of Additional Mammalian Orthologs

Because Rb and the Rb pathway are conserved between mammals and C. elegans, the powerful genetics and genomics of C. elegans can be exploited, as described herein, for the systematic identification of corresponding mammalian genes. Moreover, the comprehensive RNAi system described herein allows for the rapid identification and classification of additional genes required for cell or organism survival in the absence of Rb.

Protein sequences corresponding to genes of interest were retrieved from the repositories of C. elegans sequence information at wormbase (www.wormbase.org). The protein sequence was then used for standard [BLASTP] searching using the NCBI website (http://www.ncbi.nlm.nih.gov/BLAST/). These methods allowed us to identify mammalian orthologs of the worm genes revealed by our genetic or RNAi analysis. An ortholog is a protein that is functionally related to a reference sequence. Such orthologs might be expected to functionally substitute for one another. For example, expression of a mammalian ortholog of a C. elegans gene, when expressed in a worm having a mutation in the C. elegans gene, might be expected to partially or completely rescue the worm phenotype.

RNAi in Mammalian Cell Lines

RNAi has been used extensively to deplete mRNAs in mammalian cell culture (Elbashir et al., Nature 411:494-8, 2001). Mammalian candidate genes that are required for viability in mammalian cells lacking Rb or a functional Rb pathway are identified using RNAi, for example, in mammalian cultured cells. Briefly, an inhibitory nucleic acid is introduced into mammalian cells that contains or lacks Rb or a functional Rb pathway, for example, by lipofection. The cells lacking Rb or a functional Rb pathway are then assayed for increased levels of cell death or apoptotic cell death relative to control cells that contain Rb and a functional Rb pathway. An increased level of cell death in mammalian cells lacking Rb or a functional Rb pathway contacted with the inhibitory nucleic acid identifies the corresponding target gene as a gene required for mammalian cell survival in the absence of Rb. Methods of detecting increased levels of cell death or apoptotic cell death are standard in the art, and are described in U.S. Pat. Nos. 6,235,524; 6,509,162; 6,570,069, and 6,541,457.

The effects of RNAi of candidate genes may be evaluated in virtually any mammalian cell type (e.g., mouse embryonic fibroblasts expressing or failing to express functional Rb, the osteosarcoma Rb^{-/-} cell line SAOS-2, the Rb^{-/-} cell line C33A and the Rb^{+/+} cell line CV-1 (Dahiya et al., Mol Cell 8: 557-568, 2001; Flemington et al., Proc Natl Acad Sci U S A 90: 6914-8, 1993; Luo et al., Cell 92:463-73, 1998). While any of the mammalian homologs identified according to the methods described herein may be targeted for RNAi in mammalian cell culture, particularly promising candidates are those that are cell-autonomously synthetically lethal in the tissue-specific assays described above. In addition to cell lines that fail to express functional Rb, cell lines lacking other members of the Rb pathway may be used in the mammalian cell culture RNAi screen described above. In one example, cells lacking functional E2F family members are used. In the same way that efl-1 E2F mutant worm strains can be used to assess whether candidate gene products act in parallel to the entire Rb pathway or only to parts of it, cell lines lacking functional E2Fs could be used.

The above-described mammalian cell assays will likely identify clinically useful dsRNAs that are lethal to cells lacking Rb, but that do not affect cells expressing functional Rb and mammalian genes that are promising clinical targets for the treatment of neoplasia. In addition, the use of a two tier RNAi screen, in both C. elegans and mammalian cells, suggests that this approach will be useful for the discovery of other clinical targets in molecular pathways conserved from worms to mammals.

Microarrays

The global analysis of gene expression using gene chips can provide insights into gene expression perturbations in neoplastic tissues. Such studies can, for example, compare the expression profiles of mammalian genes of the invention, such as those listed in Tables 1 and 2, and their mammalian orthologs, whose expression is altered in neoplastic or corresponding normal tissues.

Thus, the genes listed in Tables 1 and 2, their encoded polypeptides, or fragments thereof, are useful as hybridizable array elements in a microarray. The array elements are organized in an ordered fashion such that each element is present at a specified location on the substrate. Useful substrate materials include membranes composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28:e3.i-e3.vii, 2000), MacBeath et al., (Science 289:1760-1763, 2000), Zhu et al.(Nature Genet. 26:283-289), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in PCT application W095/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample, such as a cultured cell, a tissue specimen, or other source, may be used to produce a hybridization probe as described herein. The mRNA is isolated according to standard methods, and cDNA is produced and used as a template to make complementary RNA suitable for hybridization using standard methods. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1 % SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

Families of proteins, such as those encoded by the genes listed in Tables 1 and 2 or their orthologs, may be analyzed using protein microarrays. Such arrays are useful in high-throughput low-cost screens to identify peptide or candidate compounds that-bind a polypeptide of the invention, or fragment thereof. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides encoded by a nucleic acid molecule listed in Table 1 or Table 2 or antibodies against such polypeptides) are spotted on a substrate using any convenient method known to the skilled artisan (e.g., by hand or by inkjet printer). Preferably, such methods retain the biological activity or function of the protein bound to the substrate The protein microarray is hybridized with a detectable probe. Such probes can be polypeptide, nucleic acid, or small molecules. For some applications, polypeptide and nucleic acid probes are derived from a biological sample taken from a patient, such as a a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Inhibitory Nucleic Acids

Inhibitory nucleic acids (e.g., double stranded RNA (dsRNA), short interfering RNA (siRNA), antisense RNA, and analogs thereof) interfere with the expression of a target gene. Using the nucleic acid sequence of genes listed in Tables 1 or 2, inhibitory nucleic acids that target a gene required for cell survival in the absence of Rb may be produced. Such oligonucleotide therapeutics are useful in the treatment of neoplastic diseases.

For any of the methods of application described herein, the inhibitory nucleic acid is desirably applied to a site (e.g., by injection) containing cells that-lack Rb or a functional Rb pathway (e.g., tumor cells or neoplastic cells) where cell death is to be induced. The inhibitory nucleic acid may also be applied to tissue in the vicinity of the tumor or neoplasm or to a blood vessel supplying the cells predicted to require enhanced apoptosis. Alternatively, the inhibitory nucleic acids may be administered systemically.

The inhibitory nucleic acid may be produced and isolated by any one of many standard techniques. Administration of inhibitory nucleic acid to neoplastic cells can be carried out by any of the methods for direct nucleic acid administration, as described herein. Because inhibitory nucleic acids may be substrates for nuclease degradation, modified or substituted inhibitory nucleic acids are often preferred because of properties such as, for example, enhanced cellular uptake and increased stability in the presence of nucleases. Methods for making and administering inhibitory nucleic acids or nucleic acid analogs having increased potency are standard in the art and are described herein and in published U.S. patent application Ser. Nos: 20030175950, 20030176385, 20030166282, and 20030157030.

Oligonucleotide Backbones

At least two types of oligonucleotides induce the cleavage of RNA by Rnase H: oligodeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. Although they suffer from poor membrane penetrability, Peptide Nucleic Acids (PNA) may also be employed. It is understood that the inhibitory nucleic acid methods and reagents of the present invention may be used in conjunction with any corresponding technologies that may be developed.

Locked Nucleic Acids

Locked nucleic acids (LNA) are nucleotide analogs that can be employed in the present invention. LNA contain a 2′O, 4′-C methylene bridge that restrict the flexibility of the ribofuranose ring of the nucleotide analog and locks it into the rigid bicyclic N-type conformation. LNA show improved resistance to certain exo- and endonucleases and activate RNAse H, making them suitable for use in RNAi methods. LNA can be incorporated into almost any oligonucleotide. Moreover, LNA-containing oligonucleotides can be prepared using standard phosphoramidite systhesis protocols. Additional details regarding LNA can be found in PCT publication WO99/14226, hereby incorporated by reference.

Arabinonucleic Acids

Arabinonucleic acids (ANA) can also be employed in the methods and reagents of the present invention. ANA are based on D-arabinose sugars instead of the natural D-2′-deoxyribose sugars. Underivatized ANA analogs have similar binding affinity for RNA as phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars.

siRNA

Short twenty-one to twenty-five nucleotide double stranded RNAs are effective at down-regulating gene expression in mammalian tissue culture cell lines (Elbashir et al., Nature 411:494-498, 2001, hereby incorporated by reference). Using such methods, the inactivation of mammalian orthologs may be analyzed for its effect on the survival of a neoplastic cell (e.g., a cell expressing a defective Rb nucleic acid or polypeptide). The nucleic acid sequence of a mammalian gene listed in Tables 1 and 2 can be used to design small interfering RNAs (siRNAs) that will inactivate the targeted mammalian gene for the treatment of a neoplastic disease or disease related to inappropriate cell cycle regulation.

Provided with the sequence of a mammalian target gene, siRNAs may be designed using standard methods to inactivate the gene. These siRNAs are transferred into mammalian cells in culture (e.g., neoplastic cells expressing a defective Rb nucleic acid or polypeptide), and the effect of the siRNAs on cell survival is assayed. Such methods are standard in the art and are described by Elbashir et al., (Nature 411:494-498, 2001, hereby incorporated by reference). Alternatively, siRNAs can be injected into an animal, for example, into the blood stream as described by McCaffrey et al., (Nature 418:38-9, 2002). Thus, based on the mammalian genes listed in Tables 1 and 2, oligonucleotides are designed to inhibit mammalian gene activity. Those siRNAs that are effective in reducing the survival of cultured cells (e.g., neoplastic cells) can be used in vivo as therapeutics. The injection of siRNAs corresponding to the DNA sequences of mammalian genes listed in Tables 1 and 2 would be expected to specifically inactivate those genes, thereby treating a neoplasia without damaging normal cells or causing adverse side-effects.

Screening Assays

As discussed above, the genes listed in Tables 1 and 2 are likely to be required for cell and/or organism survival in the absence of Rb. Based on this discovery, screening assays may be carried out to identify compounds that inhibit the action of a polypeptide or the expression of a nucleic acid sequence of the invention. Such compounds are useful in inducing cell death in neoplastic cells or in cells having a defect in cell cycle regulation. The method of screening may involve high-throughput techniques. In addition, these screening techniques may be carried out in cultured mammalian cells or in animals (such as nematodes or rodents).

Any number of methods are available for carrying out such screening assays. In one working example, candidate compounds are added at varying concentrations to the culture medium of cultured cells expressing one of the nucleic acid sequences of the invention. Gene expression is then measured, for example, by standard Northern blot analysis (Ausubel et al., supra) or RT-PCR, using any appropriate fragment prepared from the nucleic acid molecule as a hybridization probe. The level of gene expression in the presence of the candidate compound is compared to the level measured in a control culture medium lacking the candidate molecule. A compound that promotes a decrease in the expression of a nucleic acid sequence disclosed herein or a functional equivalent is considered useful in the invention; such a molecule may be used, for example, as a therapeutic to delay or ameliorate human diseases associated with neoplasia or inappropriate cell cycle regulation. Such cultured cells include nematode cells (for example, C. elegans cells), mammalian, or insect cells.

In another working example, the effect of candidate compounds may be measured at the level of polypeptide production using the same general approach and standard immunological techniques, such as Western blotting or immunoprecipitation with an antibody specific for a polypeptide of the invention. For example, immunoassays may be used to detect or monitor the expression of at least one of the polypeptides of the invention in an organism. Polyclonal or monoclonal antibodies (produced by standard techniques) that are capable of binding to such a polypeptide may be used in any standard immunoassay format (e.g., ELISA, Western blot, or RIA assay) to measure the level of the polypeptide. A compound that promotes a decrease in the expression of the polypeptide is considered particularly useful. Again, such a molecule may be used, for example, as a therapeutic to delay or ameliorate neoplasia.

In one example, candidate compounds may be screened for those that induce cell death in cells (e.g., neoplastic cells) or organisms (e.g., nematodes) lacking Rb or expressing a mutant Rb nucleic acid or polypeptide by targeting a gene listed in Table 1 or 2, or their mammalian orthologs. Such screens may be carried in cells in culture or in cells in vivo (e.g., in nematodes, or in a mammal, such as a rodent, having a neoplastic disorder).

In yet another working example, candidate compounds may be screened for those that specifically bind to and antagonize a polypeptide corresponding to a gene listed in Table 1 or 2, or its mammalian ortholog. The efficacy of such a candidate compound is dependent upon its ability to interact with a nucleic acid or polypeptide of the invention or a functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to decrease cell or organism survival may be assayed by any standard assay (e.g., those described herein).

In one particular working example, a candidate compound that binds to a polypeptide may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the polypeptide is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to cause cell death using any assay known to the skilled artisan. Compounds isolated by this approach may also be used, for example, as therapeutics to delay or ameliorate human diseases associated with neoplasia. Compounds that are identified as binding to polypeptides of the invention with an affinity constant less than or equal to 10 mM are considered particularly useful in the invention.

Potential antagonists include organic molecules, peptides, peptide mimetics, polypeptides, nucleic acids, and antibodies that bind to a nucleic acid sequence or polypeptide of the invention and thereby increase or decrease its activity. Potential antagonists also include small molecules that bind to and occupy the binding site of the polypeptide thereby preventing binding to cellular binding molecules, such that normal biological activity is prevented.

Each of the DNA sequences provided herein may also be used in the discovery and development of therapeutic lead compounds. The encoded protein, upon expression, can be used as a target for the screening of therapeutics for the treatment of neoplasia. Additionally, the DNA sequences encoding the amino terminal regions of the encoded protein or Shine-Delgamo or other translation facilitating sequences of the respective mRNA can be used to construct antisense, dsRNAs, or siRNA sequences to control the expression of the coding sequence of interest. Such sequences may be isolated by standard techniques (Ausubel et al., supra). The antagonists of the invention may be employed, for instance, to delay or ameliorate human diseases associated with neoplasia.

Optionally, compounds identified in any of the above-described assays may be confirmed as useful in delaying or ameliorating human diseases associated with neoplasia or inappropriate cell cycle regulation in either standard tissue culture methods or animal models and, if successful, may be used as therapeutics for the treatment of neoplasia.

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of delaying or ameliorating human diseases associated with neoplasia are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known to function in neoplasia should be employed whenever possible.

When a crude extract is found to decrease cell or organism survival, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract that decreases the survival of a cell or organism expressing a defective Rb polypeptide or nucleic acid. Methods of fractionation and purification of such heterogenous extracts are known in the art. If desired, compounds shown to be useful agents to delay or ameliorate human diseases associated with neoplasia are chemically modified according to methods known in the art.

For example, the expression of genes required in the absence of Rb is likely upregulated in neoplastic tissues lacking Rb. In one example, the expression of any one or all of the genes listed in Tables 1 and 2 is evaluated in cells or tissues lacking Rb or a member of the Rb pathway relative to cells or tissues expressing functional Rb or having a functional Rb pathway. In another example, the expression of any one or all of the genes listed in Tables 1 and 2 is evaluated in cells targeted for RNAi of a gene of interest. Optionally, the cells lack functional Rb or express a mutation in a member of the Rb pathway. Genes identified as having increased expression using such methods are expected to be genes required for the survival of cells lacking Rb or expressing a defective Rb polypeptide or nucleic acid. Such genes represent promising therapeutic targets.

Pharmaceutical Therapeutics

The invention provides a simple means for identifying compositions (including nucleic acids, peptides, small molecule inhibitors, and mimetics) capable of acting as therapeutics for the treatment of a neoplastic disease. Accordingly, a chemical entity discovered to have medicinal value using the methods described herein is useful as a drug or as information for structural modification of existing compounds, e.g., by rational drug design. Such methods are useful for screening compounds having an effect on a variety of diseases characterized by inappropriate cell cycle regulation.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a neoplastic disease therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplastic disease. Generally, amounts will be in the range of those used for other agents used in the treatment of a neoplastic disease, although in certain instances lower amounts will be needed because of the increased specificity of the compound. A compound is administered at a dosage that controls the clinical or physiological symptoms of a neoplastic disease as determined by, for example, measuring tumor size, cell proliferation, or metastasis.

Formulation of Pharmaceutical Compositions

The administration of an inhibitory nucleic acid may be by any suitable means that results in a concentration of the inhibitory nucleic acid that, combined with other components, is anti-neoplastic upon reaching the target region. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the inhibitory nucleic acid within the body over an extended period of time; (ii) formulations that after a predetermined lag time create a substantially constant concentration of the inhibitory nucleic acid within the body over an extended period of time; (iii) formulations that sustain inhibitory nucleic acid action during a predetermined time period by maintaining a relatively, constant, effective inhibitory nucleic acid level in the body with concomitant minimization of undesirable side effects associated with fluctuations in the plasma level of the active inhibitory nucleic acid substance (sawtooth kinetic pattern); (iv) formulations that localize inhibitory nucleic acid action by, e.g., spatial placement of a controlled release composition adjacent to or in the diseased tissue or organ; (v) formulations that allow for convenient dosing, such that doses are administered, for example, once every one or two weeks; and (vi) formulations that target inhibitory nucleic acid action by using carriers or chemical derivatives to deliver the inhibitory nucleic acid to a particular target cell type. Administration of inhibitory nucleic acid compounds in the form of a controlled release formulation is especially preferred for inhibitory nucleic acids having a narrow absorption window in the gastro-intestinal tract or a very short biological half-life. In these cases, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the inhibitory nucleic acid is formulated with appropriate excipients into a pharmaceutical composition that, upon administration, releases the inhibitory nucleic acid in a controlled manner. Examples include single or multiple unit tablet or capsule compositions, oil solutions, suspensions, emulsions, microcapsules, microspheres, molecular complexes, nanoparticles, patches, and liposomes.

Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active inhibitory nucleic acid(s), the composition may include suitable parenterally acceptable carriers and/or excipients. The active inhibitory nucleic acid(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

As indicated above, the pharmaceutical compositions according to the invention may be in the form suitable for sterile injection. To prepare such a composition, the suitable active inhibitory nucleic acid(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active inhibitory nucleic acid(s) may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Solid Dosage Forms For Oral Use

Formulations for oral use of inhibitory nucleic acid include tablets containing the active ingredient(s) in a mixture with non-toxic pharmaceutically acceptable excipients. Such formulations are known to the skilled artisan (e.g., 5,824,300, 5,817,307, 5,830,456, 5,846,526, 5,882,640, 5,910,304, 6,036,949, 6,036,949, 6,372,218, hereby incorporated by reference). Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active inhibitory nucleic acid substance in a predetermined pattern (e.g., in order to achieve a controlled release formulation) or it may be adapted not to release the active inhibitory nucleic acid substance until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active inhibitory nucleic acid substance). The coating may be applied on the solid dosage form in a similar manner as that described in Encyclopedia of Pharmaceutical Technology, supra.

In one example, two inhibitory nucleic acids may be mixed together in the tablet, or may be partitioned. In one example, the first inhibitory nucleic acid is contained on the inside of the tablet, and the second inhibitory nucleic acid is on the outside, such that a substantial portion of the second inhibitory nucleic acid is released prior to the release of the first inhibitory nucleic acid.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active inhibitory nucleic acid by controlling the dissolution and/or the diffusion of the active inhibitory nucleic acid substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the inhibitory nucleic acid(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Combination Therapies

Inhibitory nucleic acids may be administered in combination with any other standard cancer therapy; such methods are known to the skilled artisan (e.g., Wadler et al., Cancer Res. 50:3473-86, 1990), and include, but are not limited to, chemotherapy, radiotherapy, and any other therapeutic method used for the treatment of cancer.

Treatment of a Neoplastic Disease

The invention features therapeutic compositions and methods for treating neoplastic disease by disrupting the expression of nucleic acids required for cell survival in the absence of Rb. Interfering with the expression of such genes will likely induce cell death in neoplastic cells lacking functional Rb or a functional Rb pathway, while leaving normal cells intact. Given this specificity, therapeutics targeting the nucleic acids listed in Tables 1 or 2, or their mammalian orthologs, are expected to have few adverse effects when administered to patients than conventional chemotherapeutics.

Gene therapy is one therapeutic approach for preventing or ameliorating a neoplastic disease characterized by the loss of Rb or a functional Rb pathway. An expression vector encoding an inhibitory nucleic acid molecule (dsRNA, siRNA, or antisense RNA) targeting a gene listed in Tables 1 or 2, or a mammalian ortholog thereof, can be delivered to a neoplastic cell, such as a cell that lacks Rb expression or biological activity. The nucleic acid molecules must be delivered to such cells in a form in which they can be taken up by the cells.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a an inhibitory nucleic acid can be cloned into a retroviral vector and expression can be driven from the endogenous promoter of its corresponding gene, from the retroviral long terminal repeat, or from a promoter specific for a target cell type of interest (e.g., a neoplastic cell). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer the gene of interest (e.g., a vector encoding an inhibitory nucleic acid corresponding to a gene listed in Table 1 or Table 2) systemically or to a neoplastic cell.

Non-viral approaches can also be employed for the introduction of a therapeutic to a cell of a patient with a neoplastic disease. For example, a nucleic acid molecule can be introduced into a cell by administering the nucleic acid in the presence of lipofection (Felgner et al., Proc. Natl. Acad. Sci. U.S.A. 84:7413, 1987; Ono et al., Neuroscience Letters 17:259, 1990; Brigham et al., Am. J Med. Sci. 298:278, 1989; Staubinger et al., Methods in Enzymology 101:512, 1983), asialoorosomucoid-polylysine conjugation (Wu et al., Journal ofBiological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The dosage of the administered nucleic acid depends on a number of factors, including the size and health of the individual patient. For any particular subject, the specific dosage regimes should be adjusted over time according to the individual need and the professional judgement of the person administering or supervising the administration of the compositions. Generally, between 0.1 mg and 100 mg, is administered per day to an adult in any pharmaceutically acceptable formulation.

Combination Therapies

Therapeutics targeting a gene identified in Tables 1 or 2, or their mammalian orthologs, may be administered in combination with any other standard neoplasia therapy; such methods are known to the skilled artisan (e.g., Wadler et al., Cancer Res. 50:3473-86, 1990), and include, but are not limited to, chemotherapy, hormone therapy, immunotherapy, radiotherapy, and any other therapeutic method used for the treatment of neoplasia.

Patient Monitoring

The disease state or treatment of a patient having a neoplastic disease can be monitored using the methods and compositions of the invention. In one embodiment, a microarray is used to assay the expression profile of at least one of the nucleic acids listed in Table 1 or 2. Such monitoring may be useful, for example, in assessing the efficacy of a particular drug in a patient or in assessing patient compliance with a treatment regimen. Therapeutics that decrease the expression of at least one nucleic acid molecule or polypeptide listed in Table 1 or 2, or their mammalian orthologs, are taken as particularly useful in the invention.

Other Embodiments

From the foregoing description, it will be apparent that variations and modifications may be made to the invention described herein to adopt it to various usages and conditions. Such embodiments are also within the scope of the following claims. All publications mentioned in this specification, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

1. A method for identifying a gene required for survival in a cell having a mutation in Rb or in a component of an Rb pathway, said method comprising:
(a) providing a cell having a mutation in Rb or in a component of an Rb pathway;
(b) contacting said cell with an inhibitory nucleic acid; and
(c) comparing the phenotype of said cell contacted with said inhibitory nucleic acid with the phenotype of a control cell not contacted with said inhibitory nucleic acid, wherein an alteration in the phenotype of said cell identifies said gene as a gene that is required for survival in a cell having a mutation in Rb or in a component of an Rb pathway.

2. The method of claim 1, wherein said phenotype is associated with an alteration in apoptosis, cellular proliferation, or oncogenesis.

3. The method of claim 1, wherein said phenotype is associated with an alteration in cellular senescence, differentiation, or tumorigenesis.

4. The method of claim 1, wherein said cell has a mutation in Dp, E2F, or histone deacetylase.

5. The method of claim 1, wherein said cell is a mammalian cell.

6. The method of claim 5, wherein said cell is a human cell.

7. The method of claim 6, wherein said cell is a neoplastic cell.

8. The method of claim 2, wherein said alteration is an increase in cell death.

9. The method of claim 1, wherein said cell is a nematode cell.

10. The method of claim 7, wherein said nematode cell is in a nematode.

11. The method of claim 10, wherein said phenotype is selected from the group consisting of sterility, embryonic lethality, larval lethality, or larval growth arrest prior to L3.

12. A method for identifying a candidate compound for the treatment or prevention of a neoplasia, said method comprising:
(a) providing a cell that expresses a nucleic acid required for cell survival in the absence of Rb;
(b) contacting said cell with a candidate compound; and
(c) comparing the expression of said nucleic acid molecule in said cell contacted with said candidate compound with the expression of said nucleic acid molecule in a control cell not contacted with said candidate compound, wherein a decrease in said expression identifies said candidate compound as a candidate compound that treats a neoplasia.

13. The method of claim 12, wherein said decrease is a decrease in transcription or translation.

14. The method of claim 12, wherein said cell is in a nematode.

15. The method of claim 14, wherein said nucleic acid is selected from the group consisting of C43E11.10, M04F3.1, F28B3.7, C55B7.8, T21G5.3, F22D6.5, R05D11.7, C25A1.3, C44E4.4, C06A5.5, C37A2.4, T25G3.3, T05E8.3, D1081.8, B0511.6, Y105E8C.e, Y40B1B.6, ZC123.3, H06O01.3, K02B12.1, Y47H9C.7, Y65B4A_{—}182.c, C48E7.2, F14B4.3, W04A8.7, R12E2.12, Y106G6H.3, C43E11.9, T25G3.3, C03D6.8, T23D8.4, F46F11.4, B0207.6, C55B7.6, M05B5.2, F07A5.1, F27D4.1, F45H11.2, R06C1.3, W09C5.8, Y54E5A.4, R06A10.2, M01 B12.3, R12E2.10, F55A12.7, C01H6.7, F30A10.6, T23D8.1, Y52B11A.9, T04D3.3, Y40B1A.4, C01A2.3, Y87G2A.s, Y6B3A.1, Y54E10_{—}156.a, K07A12.2, C43H8.3, C32E8.5, T03F1.8, F57C9.4, F28B3.1, T19B4.4, C10G11.5, T21G5.4, C30F12.4, F27D4.5, C09H6.1, C34B7.3, F30A10.10, F26E4.6, F27C1.6, C55B7.9, C36B1.8, ZK858.2, ZK39.6, K08C9.1, Y34D9A_{—}151.a, Y54E10_{—}155.c, Y54E10_{—}155.e, R119.6, C10H11.10, F48C1.4, F55A12.8, ZC308.2, ZK265.6, F30A10.9, T23D8.3, Y95D11A.a, Y87G2A.e, Y71A12B.b, Y48G8A_{—}3671.a, Y65B4A_{—}174.b, ZC123.2, R12E2.2, T19B4.5, C26C6.5, F02E9.3, F39H2.3, ZK858.7, C03D6.1, Y39G10A_{—}246.I, Y39G10A_{—}246.j, Y47G6A_{—}247.h, Y48G1A_{—}54.d, and Y54E10B_{—}159.e, or an ortholog thereof.

16. The method of claim 12, wherein said cell is in a mammal.

17. The method of claim 16, wherein said nucleic acid is selected from the group consisting of CDC6, ORC1L, ORC4L, RPA2, SMC1L1, SMC1L2, DBR1, FLJ10998, DDX4, DDX3, DBY, PRP4, HIPK3, HIPK2, RY1, RNMT, SSB, LOC51068, DDX33, DDX8, DDX38, CDC5L, DDX18, KIAA0601, C20ORF16, ATBF1, SEC14L2, RNAP3, RPC62, RPO1-2, POLR2B, FLJ10388, TAF1, RPL30, HSPC031, CGI-07, LOC51068, C15ORF15, EIF3S8, UBL5, FLJ10349, SLC26A2, SLC26A8b, SLC26A8a, SLC26A1a, SLC26A1b, SLC26A1c, ETFA, UBA52, RPS27A, UBB, WASF1, COX4I1, COX4I2, MUC1, GNAS, GNAL, ARPC5, MGC3038, AP1M1, BRD7, FLJ13441, BRD1, SACM1L, SAC2, SYNJ1, FZD1, FZD7, FZD2, KIN, PDE1A, PDE1C, PDE1B, OXAOXA1L, VPS28, ARFGEF2, GUK1, KIAA0934, MCJ, FLJ10782, MSF, BCKDHB, ZNF208, CYP2A13, CYP2A7, CYP2A6, NP_{—}115612, NP_{—}001858, KIAA0266, FLJ20045, FLJ10774, HSPC111, CGI-35, LOC51077, F36A2.12, XP_{—}293124, and LOC51605.

18. A method for identifying a candidate compound for the treatment or prevention of neoplasia, said method comprising:
(a) providing a cell expressing a polypeptide encoded by a nucleic acid required for survival in a cell lacking Rb;
(b) contacting said cell with a candidate compound; and
(c) comparing the biological activity of said polypeptide in said cell contacted with said candidate compound to a control cell not contacted with said candidate compound, wherein a decrease in the biological activity of said polypeptide identifies said candidate compound as a candidate compound for the treatment or prevention of a neoplasia.

19. The method of claim 18, wherein said cell is in a nematode.

20. The method of claim 19, wherein said nucleic acid is selected from the group consisting of C43E11.10, M04F3.1, F28B3.7, C55B7.8, T21G5.3, F22D6.5, R05D11.7, C25A1.3, C44E4.4, C06A5.5, C37A2.4, T25G3.3, T05E8.3, D1081.8, B0511.6, Y105E8C.e, Y40B1B.6, ZC123.3, H06O01.3, K02B12.1, Y47H9C.7, Y65B4A_{—}182.c, C48E7.2, F14B4.3, W04A8.7, R12E2.12, Y106G6H.3, C43E11.9, T25G3.3, C03D6.8, T23D8.4, F46F11.4, B0207.6, C55B7.6, M05B5.2, F07A5.1, F27D4.1, F45H11.2, R06C1.3, W09C5.8, Y54E5A.4, R06A10.2, M01B12.3, R12E2.10, F55A12.7, C01H6.7, F30A10.6, T23D8.1, Y52B11A.9, T04D3.3, Y40B1A.4, C01A2.3, Y87G2A.s, Y6B3A.1, Y54E10_{—}156.a, K07A12.2, C43H8.3, C32E8.5, T03F1.8, F57C9.4, F28B3.1, T19B4.4, C10G11.5, T21G5.4, C30F12.4, F27D4.5, C09H6.1, C34B7.3, F30A10.10, F26E4.6, F27C1.6, C55B7.9, C36B1.8, ZK858.2, ZK39.6, K08C9.1, Y34D9A_{—}151.a, Y54E10_{—}155.c, Y54E10_{—}155.e, R119.6, C10H11.10, F48C1.4, F55A12.8, ZC308.2, ZK265.6, F30A10.9, T23D8.3, Y95D11A.a, Y87G2A.e, Y71A12B.b, Y48G8A_{—}3671.a, Y65B4A_{—}174.b, ZC123.2, R12E2.2, T19B4.5, C26C6.5, F02E9.3, F39H2.3, ZK858.7, C03D6.1, Y39G10A_{—}246.I, Y39G10A_{—}246.j, Y47G6A_{—}247.h, Y48G1A_{—}54.d, and Y54E10B_{—}159.e, or an ortholog thereof.

21. The method of claim 18, wherein said cell is in a mammal.

22. The method of claim 21, wherein said nucleic acid is selected from the group consisting of CDC6, ORC1L, ORC4L, RPA2, SMC1L1, SMC1L2, DBR1, FLJ10998, DDX4, DDX3, DBY, PRP4, HIPK3, HIPK2, RY1, RNMT, SSB, LOC51068, DDX33, DDX8, DDX38, CDC5L, DDX18, KIAA0601, C20ORF16, ATBF1, SEC14L2, RNAP3, RPC62, RPO1-2, POLR2B, FLJ10388, TAF1, RPL30, HSPC031, CGI-07, LOC51068, C15ORF15, EIF3S8, UBL5, FLJ10349, SLC26A2, SLC26A8b, SLC26A8a, SLC26A1a, SLC26A1b, SLC26A1c, ETFA, UBA52, RPS27A, UBB, WASF1, COX4I1, COX4I2, MUC1, GNAS, GNAL, ARPC5, MGC3038, AP1M1, BRD7, FLJ13441, BRD1, SACM1L, SAC2, SYNJ1, FZD1, FZD7, FZD2, KIN, PDE1A, PDE1C, PDE1B, OXAOXA1L, VPS28, ARFGEF2, GUK1, KIAA0934, MCJ, FLJ10782, MSF, BCKDHB, ZNF208, CYP2A13, CYP2A7, CYP2A6, NP_{—}115612, NP_{—}001858, KIAA0266, FLJ20045, FLJ10774, HSPC111, CGI-35, LOC51077, F36A2.12, XP_{—}293124, and LOC51605.

23. A method for identifying a candidate compound for the treatment or prevention of neoplasia, said method comprising:
(a) contacting a polypeptide encoded by a nucleic acid required for cell survival in a cell lacking Rb with a candidate compound; and
(b) detecting binding of said candidate compound to said polypeptide, wherein said binding identifies said candidate compound as a candidate compound for the treatment or prevention of neoplasia.

24. The method of claim 23, wherein said nucleic acid is selected from the group consisting of C43E 11.10, M04F3.1, F28B3.7, C55B7.8, T2 1 G5.3, F22D6.5, R05D11.7, C25A1.3, C44E4.4, C06A5.5, C37A2.4, T25G3.3, T05E8.3, D1081.8, B0511.6, Y105E8C.e, Y40B1B.6, ZC123.3, H06O01.3, K02B12.1, Y47H9C.7, Y65B4A_{—}182.c, C48E7.2, F14B4.3, W04A8.7, R12E2.12, Y106G6H.3, C43E11.9, T25G3.3, C03D6.8, T23D8.4, F46F11.4, B0207.6, C55B7.6, M05B5.2, F07A5.1, F27D4.1, F45H11.2, R06C1.3, W09C5.8, Y54E5A.4, R06A10.2, M01B12.3, R12E2.10, F55A12.7, C01H6.7, F30A10.6, T23D8.1, Y52B11A.9, T04D3.3, Y40B1A.4, C01A2.3, Y87G2A.s, Y6B3A.1, Y54E10_{—}156.a, K07A12.2, C43H8.3, C32E8.5, T03F1.8, F57C9.4, F28B3.1, T19B4.4, C10G11.5, T21G5.4, C30F12.4, F27D4.5, C09H6.1, C34B7.3, F30A10.10, F26E4.6, F27C1.6, C55B7.9, C36B1.8, ZK858.2, ZK39.6, K08C9.1, Y34D9A_{—}151.a, Y54E10_{—}155.c, Y54E10_{—}155.e, R119.6, C10H11.10, F48C1.4, F55A12.8, ZC308.2, ZK265.6, F30A10.9, T23D8.3, Y95D11A.a, Y87G2A.e, Y71A12B.b, Y48G8A_{—}3671.a, Y65B4A_{—}174.b, ZC123.2, R12E2.2, T19B4.5, C26C6.5, F02E9.3, F39H2.3, ZK858.7, C03D6.1, Y39G10A_{—}246.1, Y39G10A_{—}246j, Y47G6A_{—}247.h, Y48G1A_{—}54.d, and Y54E10B_{—}159.e, or an ortholog thereof.

25. The method of claim 23, wherein said nucleic acid is selected from the group consisting of CDC6, ORC1L, ORC4L, RPA2, SMC1L1, SMC1L2, DBR1, FLJ10998, DDX4, DDX3, DBY, PRP4, HIPK3, HIPK2, RY1, RNMT, SSB, LOC51068, DDX33, DDX8, DDX38, CDC5L, DDX18, KIAA0601, C20ORF16, ATBF1, SEC14L2, RNAP3, RPC62, RPO1-2, POLR2B, FLJ10388, TAF1, RPL30, HSPC031, CGI-07, LOC51068, C15ORF15, EIF3S8, UBL5, FLJ10349, SLC26A2, SLC26A8b, SLC26A8a, SLC26A1a, SLC26A1b, SLC26A1c ETFA, UBA52, RPS27A, UBB, WAS1, COX4I1, COX4I2, MUC1, GNAS, GNAL, ARPC5, MGC3038, AP1M1, BRD7, FLJ13441, BRD1, SACM1L, SAC2, SYNJ1, FZD1, FZD7, FZD2, KIN, PDE1A, PDE1C, PDE1B, OXAOXA1L, VPS28, ARFGEF2, GUK1, KIAA0934, MCJ, FLJ10782, MSF, BCKDHB, ZNF208, CYP2A13, CYP2A7, CYP2A6, NP_{—}115612, NP_{—}001858, KIAA0266, FLJ20045, FLJ10774, HSPC111, CGI-35, LOC51077, F36A2.12, XP_{—}293124, and LOC51605.
